SEQUENCE LISTING (29 sequences)

SEQ ID NO: 1 - RNA, 21 nt, Artificial Sequence
  Description of Artificial Sequence Synthetic oligonucleotide
gaaaaucgcuuauauaccuuu

SEQ ID NO: 2 - RNA, 21 nt, Artificial Sequence
  Description of Artificial Sequence Synthetic oligonucleotide
agguauauaagcgauuuucuu

SEQ ID NO: 3 - RNA, 21 nt, Artificial Sequence
  Description of Artificial Sequence Synthetic oligonucleotide
ugaaguugggcaugacuaauu

SEQ ID NO: 4 - RNA, 21 nt, Artificial Sequence
  Description of Artificial Sequence Synthetic oligonucleotide
uuagucaugcccaacuucauu

SEQ ID NO: 5 - RNA, 21 nt, Artificial Sequence
  Description of Artificial Sequence Synthetic oligonucleotide
ggacccaugucuucggaaauu

SEQ ID NO: 6 - RNA, 21 nt, Artificial Sequence
  Description of Artificial Sequence Synthetic oligonucleotide
uuuccgaagacauggguccuu

SEQ ID NO: 7 - RNA, 20 nt, Artificial Sequence
  Description of Artificial Sequence Synthetic oligonucleotide
ggagaaugcaaauauguauu

SEQ ID NO: 8 - RNA, 21 nt, Artificial Sequence
  Description of Artificial Sequence Synthetic oligonucleotide
ucacauauuugcauucuccuu

SEQ ID NO: 9 - RNA, 21 nt, Artificial Sequence
  Description of Artificial Sequence Synthetic oligonucleotide
ugauaacgaaccacaaauauu

SEQ ID NO: 10 - RNA, 21 nt, Artificial Sequence
  Description of Artificial Sequence Synthetic oligonucleotide
uauuugugguucguuaucauu

SEQ ID NO: 11 - RNA, 21 nt, Artificial Sequence
  Description of Artificial Sequence Synthetic oligonucleotide
ugagugaaaugccuucuaguu

SEQ ID NO: 12 - RNA, 21 nt, Artificial Sequence
  Description of Artificial Sequence Synthetic oligonucleotide
cuagaaggcauuucacucauu

SEQ ID NO: 13 - RNA, 21 nt, Artificial Sequence
  Description of Artificial Sequence Synthetic oligonucleotide
guuauuacaguccagcuuauu

SEQ ID NO: 14 - RNA, 21 nt, Artificial Sequence
  Description of Artificial Sequence Synthetic oligonucleotide
uaagcuggacuguaauaacuu

SEQ ID NO: 15 - RNA, 21 nt, Artificial Sequence
  Description of Artificial Sequence Synthetic oligonucleotide
gaaagaaacuucgacaagauu

SEQ ID NO: 16 - RNA, 21 nt, Artificial Sequence
  Description of Artificial Sequence Synthetic oligonucleotide
ucuugucgaaguuucuuucuu

SEQ ID NO: 17 - DNA, 36 bp, Artificial Sequence
  Description of Artificial Sequence Synthetic oligonucleotide
ttttttggatccaatgacacctactctgggaagcgt

SEQ ID NO: 18 - DNA, 35 bp, Artificial Sequence
  Description of Artificial Sequence Synthetic oligonucleotide
ttttttaagcttaattttttccatttttgcctccc

SEQ ID NO: 19 - DNA, 36 bp, Artificial Sequence
  Description of Artificial Sequence Synthetic oligonucleotide
ttttttggatccctcggctcaggccattatgctgct

SEQ ID NO: 20 - DNA, 32 bp, Artificial Sequence
  Description of Artificial Sequence Synthetic oligonucleotide
ttttttaagctttacctgttcaactctgactg

SEQ ID NO: 21 - DNA, 26 bp, Artificial Sequence
  Description of Artificial Sequence Synthetic oligonucleotide
tttctggctgcagttctctcggcact

SEQ ID NO: 22 - DNA, 33 bp, Artificial Sequence
  Description of Artificial Sequence Synthetic oligonucleotide
cctctcctatggtacctaaacatgagaagcccc

SEQ ID NO: 23 - DNA, 20 bp, Artificial Sequence
  Description of Artificial Sequence Synthetic oligonucleotide
accacagtccatgccatcac

SEQ ID NO: 24 - DNA, 20 bp, Artificial Sequence
  Description of Artificial Sequence Synthetic oligonucleotide
tccaccaccctgttgctgta

SEQ ID NO: 25 - DNA, 31 bp, Artificial Sequence
  Description of Artificial Sequence Synthetic oligonucleotide
atctggcaccacacctttacaatgagctgcg

SEQ ID NO: 26 - DNA, 32 bp, Artificial Sequence
  Description of Artificial Sequence Synthetic oligonucleotide
cgtcatactcctgcttgctgatccacatctgc

SEQ ID NO: 27 - protein, 168 aa, Mus sp.
MECNWILPFILSVTSGVYSQVQLQQSGAELARPGASVKLSCKASGYTFTRYWMQWIKQRPGQGLEWIGAIYPGNGDIRYTQKFKGKATLTADKSSSTAYMQLSSLASEDSAVYYCARGTTPSSYWGQGTLVTVSAAKTTAPSVYPLAPVCGDTTGSSVTLGCLVKGYF

SEQ ID NO: 28 - protein, 167 aa, Mus sp.
MMSPAQFLFLLVLWIRETSGDVVMTQTPLTLSVSIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIKRADAAPTVSIFPPSSEQLTSGGASVVCFLNNFYPK

SEQ ID NO: 29 - protein, 6 aa, Artificial Sequence
  Description of Artificial Sequence Synthetic 6xHis tag
HHHHHH

(19) United States
(12) Patent Application Publication
(10) Pub. No.: US 2010/0111965 A1
(43) Pub. Date: May 6, 2010

(54) COMBINATION THERAPY

(75) Inventors: James Johnston, Belfast (GB); Shane Olwill, Belfast (GB); Jill Brown, Belfast (GB); Nuala Morgan, Belfast (GB); Thomas Jaquin, Belfast (GB); Christopher Scott, Belfast (GB)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: FUSION ANTIBODIES LIMITED, BELFAST, NI (GB)

(21) Appl. No.: 12/445,109

(22) PCT Filed: Oct. 11, 2007
(86) PCT No.: PCT/GB2007/050623

§ 371 Date: Dec. 7, 2009

Related U.S. Application Data
(60) Provisional application No. 60/995,143, filed on Sep. 25, 2007.

(30) Foreign Application Priority Data
Oct. 11, 2006 (GB) 0620147.9
Nov. 2, 2006 (GB) 0621848.1
Jun. 9, 2007 (GB) 0711226.1
Jun. 9, 2007 (GB) 0711228.7

(51) Int. Cl. A61K 39/395; A61K 31/7088; C07K 16/00; A61P 35/00; C12Q 1/68

(52) U.S. Cl. 424/158.1; 514/44 R; 514/44 A; 530/387.1; 435/6

(57) ABSTRACT

The invention provides a method of treating neoplastic disease in a subject, said method comprising the simultaneous, sequential or separate, administration to said subject of an effective amount of (i) an inhibitor of a first EGF, e.g. HB-EGF and (ii) an inhibitor of a second EGF, e.g. AREG. Also described are novel synergistic combinations of EGF inhibitors with topoisomerase inhibitors which attenuate tumour cell growth. Further described are novel anti AREG antibodies.

### FIELD OF THE INVENTION

The present invention relates to cancer treatment. In particular it relates to methods of determining susceptibility to resistance to anti-cancer, drugs, methods for overcoming such resistance and combination therapies for the treatment of cancer.

### BACKGROUND TO THE INVENTION

Cancer is the leading cause of mortality in the Western countries. A large number of chemotherapeutic agents have been developed over the last 50 years to treat cancers. The majority of chemotherapeutic agents can be divided into: alkylating agents, antimetabolites, anthracyclines, plant alkaloids, topoisomerase inhibitors, and antitumour agents. All of these drugs affect cell division or DNA synthesis and function in some way.

The effectiveness of particular chemotherapeutic agents varies between cancers, between patients and over time in individual patients. Cancerous cells exposed to a chemotherapeutic agent may develop resistance to such an agent, and quite often cross-resistance to several other antineoplastic agents as well. Moreover, the narrow therapeutic index of many chemotherapeutic agents further limits their use. Accordingly, it is often necessary to change treatments of patients with cancer if the first or second line therapy is not sufficiently effective or ceases to be sufficiently effective. In many cases combinations of particular treatments have been found to be particularly effective.

For example, colorectal cancer is one of the most currently diagnosed cancers in Europe and one with the poorest 5 year survival rates. For more than 40 years, inhibitors of thymidylate synthase, for example 5-Fluorouracil (5-Fu), have been the treatment of choice for this cancer. Thymidylate synthase inhibitors act by causing DNA damage due to misincorporation of FUTP into RNA and DNA (Longley et al Nat Rev Cancer, 3:330-338, 2003; Backus et al Oncol research 2000; 12(5):231-9). More recently new chemotherapeutic agents have been introduced to the clinic, for example the topoisomerase I inhibitors (e.g. irinotecan: CPT-11) and DNA damaging agents (e.g. oxaliplatin: alkylating agents).

These chemotherapeutics agents, 5-Fu included, can be used alone but it is common that clinical regimes incorporate a combination. Indeed combined chemotherapy has shown promising results by improving the response rates in patients by acting on the tumors through different pathways. Nevertheless many patients still cannot be treated through these regimes because of drug resistance either acquired or inherent. In vitro and in vivo studies have demonstrated that increased TS expression correlates with increased resistance to 5-FU (Johnston et al, Cancer Res., 52: 4306-4312, 1992). Other upstream determinants of 5-FU chemosensitivity include the 5-FU-degrading enzyme dihydropyrimidine dehydrogenase and 5-FU-anabolic enzymes such as orotate phosphoribosyl transferase (Longley et al Nat Rev Cancer, 3:330-338, 2003).

The use of antimetabolites e.g. tomudex (TDX) and platinum containing compounds e.g. oxaliplatin is similarly limited by resistance.

Further, the choice of chemotherapy is further complicated by cancer type and, for example, whether or not the cancer is associated with a p53 mutation. For example, as described in WO2005/053739, whereas the combination of platinum based chemotherapeutics with antiFas antibodies was shown to have a synergistic cytotoxic effect in tumours with wild type p53, such synergy was not seen in p53 mutant cells.

5-Fu, CPT-11 and oxaliplatin remain front line therapies, but the development of non responsive tumours or chemotherapy resistant cancer remains a major obstacle to successful chemotherapy. Due to the importance of early treatment of cancers, there is a clear need for tools which enable prediction of whether a particular therapy, either single or combination, will be effective against particular tumours in individual patients. Moreover, there remains the need for new treatment regimes to increase the repertoire of treatments available to the physician.

### SUMMARY OF THE INVENTION

The present inventors have investigated proteins upregulated in response to treatment with different classes of chemotherapy and have surprisingly shown that a variety of genes encoding peptide growth factors of the Epidermal Growth Factor (EGF) family are overexpressed in a number of different tumour cell line models of cancer, from a number of different types of cancer, following in vivo challenge with different physiologically relevant doses of different classes of chemotherapy.

Further investigation by the inventors has surprisingly shown that combinations of inhibitors of different EGFs results in a surprisingly dramatic reduction in tumour cell growth and proliferation compared to the reduction when inhibitors of a single EGF were tested.

Accordingly, in a first aspect of the present invention, there is provided a method of treating neoplastic disease in a subject, said method comprising the simultaneous, sequential or separate, administration to said subject of an effective amount of (i) an inhibitor of a first EGF and (ii) an inhibitor of a second EGF, wherein said first and second EGF are different EGFs.

In a second aspect of the invention, the invention provides a pharmaceutical composition comprising (i) an inhibitor of a first EGF and (ii) an inhibitor of a second EGF, wherein said first and second EGFs are different EGFs.

A third aspect of the invention provides kit comprising, in combination for simultaneous, separate, or sequential use in the treatment of neoplastic disease,

(i) an inhibitor of a first EGF and
(ii) an inhibitor of a second EGF, wherein said first and second EGF are different EGFs.

Any EGF may be used in the first, second or third aspects of the invention. Thus the first and second EGFs may each be independently selected from the group consisting of HB-EGF, AREG, TGF, EREG, BTC, NRG 1, NRG2, NRG3, and NRG4.

In one embodiment, the first EGF is HB-EGF and said second EGF is selected from the group consisting of AREG, TGF, EREG, BTC, and NRG3.

In a particular embodiment, the first EGF is HB-EGF and said second EGF is AREG.

Any inhibitors of an EGF may be used. EGF inhibitors which may be used in the present invention include any molecule which reduces expression of the gene encoding the EGF or antagonizes the EGF protein. Such molecules may include, but are not limited to, antibodies, antibody fragments, immunoconjugates, small molecule inhibitors, peptide inhibitors, specific binding members, non-peptide small organic molecules, nucleic acid molecules which inhibit EGF expression, such as siRNA, antisense molecules or oligonucleotide decoys.

In one embodiment, the inhibitors of said first and second EGFs are different. In a particular embodiment, the inhibitor of the first EGF is not an inhibitor of the second EGF and vice versa.

In one embodiment of, the invention, the inhibitor of each EGF is a specific inhibitor of that EGF, i.e. not an inhibitor of another EGF.

In one embodiment, said inhibitor of said first EGF is an antibody which binds said first EGF or a nucleic acid molecule which inhibits EGF expression.

As described above, in one embodiment, said first EGF is HB-EGF.

In one such embodiment the inhibitor of the first EGF is an siRNA having sense and antisense sequences shown as Sequence ID Nos 1 and 2 respectively:

Sequence ID No: 1:
GAAAAUCGCUUAUAUACCUUU
Sequence ID No: 2:
AGGUAUAUAAGGGAUUUUCUU

In another such embodiment the inhibitor of the HB-EGF is an siRNA having sense and antisense sequences shown as Sequence ID Nos 3 and 4 respectively:

Sequence ID No: 3:
UGAAGUUGGGCAUGACUAAUU
Sequence ID No: 4:
UUAGUCAUGGCCAACUUCAUU

In another such embodiment the inhibitor of the HB-EGF is an siRNA having sense and antisense sequences shown as Sequence ID Nos 5 and 6 respectively:

Sequence ID No: 5:
GGACCCAUGUCUUCGGAAAUU
Sequence ID No: 6:
UUUCCGAAGACAUGGGUCCUU

In another such embodiment the inhibitor of the HB-EGF is an siRNA having sense and antisense sequences shown as Sequence ID Nos 7 and 8 respectively:

Sequence ID No: 7:
GGAGAAUGCAAAUAUGUAUU
Sequence ID No: 8:
UCACAUAUUUGCAUUCUCCUU

In one embodiment, the inhibitor of HB -EGF comprises a pool of two, three or four of the siRNA molecules (wherein each molecule comprises the sense and complementary antisense molecule) shown above i.e. two, three or four of the sense/antisense pairs selected from the group consisting of Sequence ID No: 1/Sequence ID No: 2, Sequence ID No: 3/Sequence ID No: 4, Sequence ID No: 5/Sequence ID No: 6, and Sequence ID No: 7/Sequence ID No: 8.

In one embodiment, said inhibitor of said second EGF is an antibody which binds said second EGF or a nucleic acid molecule which inhibits EGF expression.

In one embodiment, said second EGF is AREG. In such an embodiment, an antibody which may be used as the inhibitor of AREG is the anti-AREG antibody 6E11 1E9 106. The VH and VL sequences of the 6E11 1E9 106 antibody have been determined by the inventors and are described infra.

In one embodiment of the invention, siRNA molecules which may be used in the invention as an inhibitor of AREG is an siRNA having sense and antisense sequences shown as Sequence ID Nos 9 and 10 respectively:

Sequence ID No: 9
UGAUAACGAACCACAAAUAUU
Sequence ID No: 10
UAUUUGUGGUUCGUUAUCAUU

In another such embodiment the inhibitor of AREG is an siRNA having sense and antisense sequences shown as Sequence ID Nos 11 and 12 respectively:

Sequence ID No: 11
UGAGUGAAAUGCCUUCUAGUU
Sequence ID No: 12
CUAGAAGGCAUUUCACUCAUU

In another such embodiment the inhibitor of AREG is an siRNA having sense and antisense sequences shown as Sequence ID Nos 13 and 14 respectively:

Sequence ID No: 13
GUUAUUACAGUCCAGCUUAUU
Sequence ID No: 14
UAAGCUGGACUGUAAUAACUU

In another such embodiment the inhibitor of AREG is an siRNA having sense and antisense sequences shown as Sequence ID Nos 15 and 16 respectively:

Sequence ID No: 15
GAAAGAAACUUCGACAAGAUU
Sequence ID No: 16
UCUUGUCGAAGUUUCUUUCUU

In one embodiment, the inhibitor of AREG comprises a pool of two, three or four of the siRNA molecules (wherein each molecule comprises the sense and complementary antisense molecule) shown above i.e. two, three or four of the sense/antisense pairs selected from the group consisting of Sequence ID No: 9/Sequence ID No: 10, Sequence ID No: 11/Sequence ID No: 12, Sequence ID No: 13/Sequence ID No: 14, and Sequence ID No: 15/Sequence ID No: 16.

As described in the Examples, the inventors have developed the antibodies with specificity for AREG, which may be used in the invention. The antibodies have found to be particularly efficacious.

Accordingly, in a fourth aspect of the invention, there is provided an antibody molecule having binding specificity for AREG, wherein the antibody molecule is the 6E11 1E9 106 antibody, or a fragment thereof.

The VH and VL domain sequences of the 6E11 1E9 106 antibody has been determined by the inventor and are as follows.

6E11 1E9 1C6 VH sequence (Sequence ID No: 27):
MECNWILPFILSVTSGVYSQVQLQQSGAELARPGASVKLSCKASGYTFTR
YWMQWIKQRPGQGLEWIGAIYPGNGDIRYTQKFKGKATLTADKSSSTAYM
QLSSLASEDSAVYYCARGTTPSSYWGQGTLVTVSAAKTTAPSVYPLAPVC
GDTTGSSVTLGCLVKGYF
6E11 1E9 1CG VL sequence (Sequence ID No: 28)
MMSPAQFLFLLVLWIRETSGDVVMTQTPLTLSVSIGQPASISCKSSQSLL
DSDGKTYLNWLLQRPGQSPKRLIYLVSKLDSGVPDRFTGSGSGTDFTLKI
SRVEAEDLGVYYCWQGTHFPWTFGGGTKLEIKRADAAPTVSIFPPSSEQL
TSGGASVVCFLNNFYPK

Thus, in one embodiment of the invention the antibody molecule having binding specificity for AREG of and for use in the invention is an antibody molecule comprising at least one of the CDRs of the 6E11 1E9 106 VH region and/or at least one of the CDRs of the 6E11 1E9 106 VL region. In one embodiment, the antibody molecule comprises all three of the CDRS of the 6E11 1E9 106 VH region and/or all three of the CDRS of the 6E11 1E9 106 VL region.

In one embodiment, the specific binding member comprises an antibody variable domain (which may be VH or VL) having the VH domain sequence shown above, or an antibody variable domain (which may be VL or VH) having the antibody VL domain sequence shown above, or both.

The antibody molecule may be a whole antibody. In one alternative embodiment, the antibody molecule may be an antibody fragment such as an scFv.

The provision of the antibody molecules of the present invention enables the development of related antibodies which also inhibit tumour cell growth and which optionally have similar or greater binding specificity.

Accordingly, further encompassed within the scope of this aspect of the present invention are antibody molecules comprising an antibody variable domain (VH or VL) having the 6E11 1E9 106 VH sequence shown above in which 5 or less, for example 4, 3, 2, or 1 amino acid substitutions have been made in at least one CDR and wherein the specific binding member retains the ability to inhibit the tumour cell growth. Also encompassed by the invention are antibody molecules comprising an antibody variable domain (VL or VH) having the 6E11 1E9 106 VL sequence shown above in which 5 or less, for example 4, 3, 2, or 1 amino acid substitutions have been made in at least one CDR and wherein the specific binding member retains the ability to inhibit the tumour cell growth.

The method of any one of the first to third aspects of the invention may further comprise the simultaneous, sequential or separate, administration to said subject of an effective amount of (iii) a chemotherapeutic agent.

In one embodiment, the chemotherapeutic agent is selected from the group consisting of antimetabolites, topoisomerase inhibitors, alkylating agents, anthracyclines, and plant alkaloids.

The inventors have further shown that particular combinations of EGF inhibitors with topoisomerase inhibitors attenuate tumour cell growth to an extent greater than could be predicted from the effects of each inhibitor alone.

Accordingly, in a sixth aspect of the invention, there is provided a method of treating neoplastic disease in a subject, said method comprising the simultaneous, sequential or separate, administration to said subject of an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

In a seventh aspect of the invention, there is provided a pharmaceutical composition for the treatment of cancer, said composition comprising an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

An eighth aspect of the invention provides comprising, in combination for simultaneous, separate, or sequential use in the treatment of neoplastic disease, an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

In one embodiment of any one of the sixth, seventh or eighth aspects of the invention, the EGF is HB-EGF. In another embodiment, the EGF is AREG.

In these aspects of the invention, any topisomerase inhibitor may be used. In a particular embodiment, the topoisomerase inhibitor is CPT-11. In another embodiment, the topoisomerase inhibitor is an active metabolite of CPT-11, for example SN-38.

In one embodiment, wherein the EGF is AREG, the EGF inhibitor is the anti-AREG antibody 6E11 1E9 106.

The methods of the invention may be used to treat any neoplastic disease. In a particular embodiment, the neoplastic disease is cancer. For example, neoplastic diseases which may be treated using the compositions and methods of the invention include, but are not limited to, colorectal cancer, breast cancer, lung cancer, prostate cancer, hepatocellular cancer, lymphoma, leukaemia, gastric cancer, pancreatic cancer, cervical cancer, ovarian cancer, liver cancer, renal cancer, thyroid cancer, melanoma, carcinoma, head and neck cancer, and skin cancer.

In one particular embodiment, the neoplastic disease is colorectal cancer.

In another embodiment, the neoplastic disease is breast cancer.

In another embodiment, the neoplastic disease is lung cancer.

As described in the Examples, the inventors have shown that certain EGFs are upregulated by chemotherapies in p53 mutant tumour cells as well as in p53 wild type tumours. This is particularly surprising given that resistance to chemotherapy has previously been shown to be largely dependent on p53 status.

In a particular embodiment, the neoplastic disease is a cancer comprising a p53 mutation.

Further provided by the invention in a ninth aspect is a method of inducing and/or enhancing expression of a gene encoding an EGF protein in a cell or tissue; said method comprising administration of a topoisomerase inhibitor to said cell or tissue, wherein said EGF is selected from the group consisting of AREG, TGF, EREG, BTC, and NRG3.

The demonstration by the present inventors that expression of EGFs are upregulated in response to treatment with diverse topoisomerase inhibitors suggests that the therapeutic effect of treatment with these chemotherapies may, in certain patients, be compromised by the upregulation of EGFs.

Thus, the invention may be used in assays to determine whether or not treatment with a topoisomerase inhibitor e.g. CPT-11 or an analogue thereof may be effective in a particular patient.

Thus, in a tenth aspect of the present invention, there is provided an in vitro method for evaluating the response of tumour cells from a subject to the presence of a topoisomerase inhibitor to predict response of the tumour cells in vivo to treatment with the topoisomerase inhibitor, which method comprises:

(a) providing a sample of tumour cells from a subject;
(b) exposing a portion of said sample of tumour cells to said topoisomerase inhibitor;
(c) comparing expression of one or more genes encoding one or more EGFs wherein said EGF is selected from the group consisting of AREG, TGF, EREG, BTC, and NRG3 in said portion of the sample of tumour cells exposed to said topoisomerase inhibitor with expression of said gene(s) in a control portion of said sample which has not been exposed to said topoisomerase inhibitor; wherein enhanced expression in the portion of sample exposed to said topoisomerase inhibitor is indicative of decreased sensitivity to said topoisomerase inhibitor.

The invention further represents a tool for prognosis and diagnosis of a subject afflicted with a tumour. For the purpose of prognosis, determining the expression level of a gene before and after chemotherapeutic treatment would identify if the subject will respond to a combinatory treatment approach. For the purpose of diagnosis the expression profile of a tumours genetic response to chemotherapy would identify which combination therapy would be most effective for that tumour.

Thus, an eleventh aspect of the invention provides a method of prognosis for evaluating the response of a patient to combination therapy comprising a topoisomerase inhibitor and an inhibitor of an EGF, said method comprising (a) determining expression of a gene encoding an EGF in an in vitro sample containing tumour cells obtained from a subject prior to treatment with said chemotherapeutic treatment

(b) determining expression of said gene encoding said EGF, wherein said EGF is selected from the group consisting of AREG, TGF, EREG, BTC, and NRG3, in an in vitro sample containing tumour cells obtained from a subject after treatment with said chemotherapeutic treatment;
(c) comparing expression in (b) with expression in (a), wherein enhanced expression in (b) compared to (a) is indicative that the patient may benefit from combination therapy comprising a topoisomerase inhibitor and an inhibitor of said EGF.

In the tenth or eleventh aspects of the invention the expression of gene(s) encoding one or more EGFs may be determined. For example, the expression of genes encoding at least two, for example three, four or five of AREG, TGF, EREG, BTC, and NRG3 may be determined.

In another embodiment of the tenth or eleventh aspects of the invention the expression of genes encoding at least two, for example three, or four of TGF, EREG, BTC, and NRG3 may be determined.

In aspects of the invention involving the determination of expression of a gene encoding an EGF, the expression of any gene encoding said EGF in the subject may be determined.

For example, in an embodiment in which the EGF is AREG, the gene may be NM_{—}001657. In an embodiment, in which the EGF is HB-EGF, the gene may be NM_{—}001945.

In an embodiment of the invention, expression of said gene in the sample exposed to said chemotherapeutic agent is considered to be enhanced if the expression is at least 1.5-fold, preferably at least 2-fold, more preferably at least 5-fold, that of the one or more genes in the control portion of said sample which has not been exposed to said chemotherapeutic agent.

In the present application, unless the context demands otherwise, where reference is made to a chemotherapeutic agent and an EGF modulator, the chemotherapeutic agent and the EGF modulator are different agents. Generally, the chemotherapeutic agent will have a different mode of action from the EGF modulator. In one embodiment, the chemotherapeutic agent will not inhibit the EGF.

In a further aspect of the invention, there is provided the use of an inhibitor of a first EGF in the preparation of a medicament for simultaneous, separate or sequential use with an inhibitor of a second EGF for the treatment of neoplastic disease; wherein said first and second EGFs are different EGFs.

Another aspect of the invention provides the use of an inhibitor of a second EGF in the preparation of a medicament for simultaneous, separate or sequential use with an inhibitor of a first EGF for the treatment of neoplastic disease; wherein said first and second EGFs are different EGFs.

Another aspect which is provided is the use of an inhibitor of an EGF,

wherein said inhibitor is a nucleic acid molecule which inhibits EGF expression or an anti EGF antibody, and wherein said EGF is HB-EGF or AREG, in the preparation of a medicament for the simultaneous, separate or sequential use with a topoisomerase inhibitor in the treatment of a neoplastic disease.

Further provided is the use of a topoisomerase inhibitor in the preparation of a medicament for simultaneous, separate or sequential use with an inhibitor of an EGF in the treatment of a neoplastic disease,

wherein said inhibitor of an EGF is a nucleic acid molecule which inhibits EGF expression or an anti EGF antibody, and wherein said EGF is HB-EGF or AREG.

Preferred and alternative features of each aspect of the invention are as for each of the other aspects muatis mutandis unless the context demands otherwise.

### DETAILED DESCRIPTION

As described above and in the Examples, the present invention is based on the demonstration that expression of various EGF genes and proteins are upregulated in tumour cells in the presence of certain chemotherapies and that particular combinations of EGF inhibitors and chemotherapeutic agents as well as particular combinations of two or more EGF inhibitors demonstrate superadditive effects in the attenuation of tumour cell growth.

### Assays

As described above, in one embodiment, the present invention relates to methods of screening samples comprising tumour cells for expression of EGF genes in order to determine suitability for treatment using particular chemotherapeutic agents.

The methods of the invention may involve the determination of expression of any gene encoding an EGF. The EGF-family of peptide growth factors is made up of 10 members which have the ability to selectively bind the ErrB receptors (ErrB1 or EGF receptor, ErrB2 or Her2, ErrB3 and ErrB4).

In one embodiment of the invention, the EGF is a ligand of ErbB-1, for example, amphiregulin (AREG), TGF, Epiregulin (EREG) or BTC.

In another embodiment, the EGF is a ligand of ErbB-4, for example NRG3

Accession details are provided for each of these genes below.

| Gene | Accession No |
|---|---|
| BTC | NM_001729 |
| HB-EGF | : NM_001945 |
| AREG | NM_001657 |
| TGFA | NM_003236 |
| EREG | NM_001432 |
| NRG3 | NM_001010848 |

The expression of any gene encoding an EGF of interest may be determined.

For example, where the EGF is AREG, the Areg gene may be NM_{—}001657.

In a particular embodiment of the invention, the gene is Areg having accession no: NM_{—}001657. In another particular embodiment of the invention, the gene is the HB-EGF gene having accession no: NM_{—}001945.

The expression of each gene may be measured using any technique known in the art. Either mRNA or protein can be measured as a means of determining up- or down regulation of expression of a gene. Quantitative techniques are preferred. However semi-quantitative or qualitative techniques can also be used. Suitable techniques for measuring gene products include, but are not limited to, SAGE analysis, DNA microarray analysis, Northern blot, Western blot, immunocytochemical analysis, and ELISA.

In the methods of the invention, RNA can be detected using any of the known techniques in the art. Preferably an amplification step is used as the amount of RNA from the sample may be very small. Suitable techniques may include RT-PCR, hybridisation of copy mRNA (cRNA) to an array of nucleic acid probes and Northern Blotting.

For example, when using mRNA detection, the method may be carried out by converting the isolated mRNA to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of gene expression products of one or more genes encoding Areg in the sample. Analysis may be accomplished using Northern Blot analysis to detect the presence of the gene products in the amplification product. Northern Blot analysis is known in the art. The analysis step may be further accomplished by quantitatively detecting the presence of such gene products in the amplification products, and comparing the quantity of product detected against a panel of expected values for known presence or absence in normal and malignant tissue derived using similar primers.

Primers for use in methods of the invention will of course depend on the gene(s), expression of which is being determined. In one embodiment of the invention, one or more of the following primer sets may be used:

(SEQ ID No: 17)

| | |
|---|---|
| Forward: | TTTTTTGGATCCAATGACACCTACTCTGGGAAGCGT |

(SEQ ID No: 18)

| | |
|---|---|
| Reverse: | TTTTTTAAGCTTAATTTTTTCCATTTTTGCCTCCC |

And Exon Spanning

(SEQ ID No: 19)

| | |
|---|---|
| Forward: | TTTTTTGGATCCCTCGGCTCAGGCCATTATGCTGCT |

(SEQ ID No: 20)

| | |
|---|---|
| Reverse: | TTTTTTAAGCTTTACCTGTTCAACTCTGACTG |

(SEQ ID No: 21)

| | |
|---|---|
| Forward | 5′-TTTCTGGCTGCAGTTCTCTCGGCACT-3′ |

(SEQ ID No: 22)

| | |
|---|---|
| Reverse | 5′-CCTCTCCTATGGTACCTAAACATGAGAAGCCCC-3′ |

In e.g. determining gene expression in carrying out methods of the invention, conventional molecular biological, microbiological and recombinant DNA techniques known in the art may be employed. Details of such techniques are described in, for example, Current Protocols in Molecular Biology, 5th ed., Ausubel et al. eds., John Wiley & Sons, 2005 and, Molecular Cloning: a Laboratory Manual: 3^{rd} edition Sambrook et al., Cold Spring Harbor Laboratory Press, 2001.

The assays of the invention may be used to monitor disease progression, for example using biopsy samples at different times. In such embodiments, instead of comparing the expression of EGF against a control sample which has not been exposed to said chemotherapeutic agent, the expression of the EGF is compared against a sample obtained from the same tissue at an earlier time point, for example from days, weeks or months earlier.

The methods of the invention may be used to determine the suitability for treatment of any suitable cancer with a chemotherapeutic agent e.g. CPT-11 or analogues thereof. For example the methods of the invention may be used to determine the sensitivity or resistance to treatment of cancers including, but not limited to, gastrointestinal, such as colorectal, te, head and neck cancers.

In a particular embodiment of the invention, the methods of the invention may be used to determine the sensitivity or resistance to treatment of colorectal cancer.

In another particular embodiment of the invention, the methods of the invention may be used to determine the sensitivity or resistance to treatment of lung cancer.

In another particular embodiment of the invention, the methods of the invention may be used to determine the sensitivity or resistance to treatment of breast cancer.

The nature of the tumour or cancer will determine the nature of the sample which is to be used in the methods of the invention. The sample may be, for example, a sample from a tumour tissue biopsy, bone marrow biopsy or circulating tumour cells in e.g. blood. Alternatively, e.g. where the tumour is a gastrointestinal tumour, tumour cells may be isolated from faeces samples. Other sources of tumour cells may include plasma, serum, cerebrospinal fluid, urine, interstitial fluid, ascites fluid etc.

For example, solid tumour samples collected in complete tissue culture medium with antibiotics. Cells may be manually teased from the tumour specimen or, where necessary, are enzymatically disaggregated by incubation with collagenase/DNAse and suspended in appropriate media containing, for example, human or animal sera.

In other embodiments, biopsy samples may be isolated and frozen or fixed in fixatives such as formalin. The samples may then be tested for expression levels of genes at a later stage.

In determining treatment, it may e desirable to determine p53 status of a cancer. For example, p53 status may be useful as it may dictate the type of chemotherapy which should be used in combination with particular EGF proteins. p3 status may be determined using conventional methods. For example, the use of immunohistochemistry may be used to identify hotspot mutations while gene sequencing or other DNA analysis methodologies may also be employed. This analysis may suitably be performed on isolated tumour tissue.

### Chemotherapeutic Agents

Chemotherapeutic agents may be used in certain embodiments of the present invention. For example agents which may be used include antimetabolites, including thymidylate synthase inhibitors, nucleoside analogs, platinum cytotoxic agents, topoisomerase inhibitors or antimicrotubules agents. Examples of thymidylate synthase inhibitors which may be used in the invention include 5-FU, MTA and TDX. An example of an antimetabolite which may be used is tomudex (TDX). Examples of platinum cytotoxic agents which may be used include cisplatin and oxaliplatin.

Chemotherapeutic agents which may be used in the present invention in addition or instead of the specific agents recited above, may include alkylating agents; alkyl sulfonates; aziridines; ethylenimines; methylamelamines; nitrogen mustards; nitrosureas; anti-metabolites; folic acid analogues; purine analogs; pyrimidine analogs; androgens; anti-adrenals; folic acid replenishers; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; ionidamine; mitoguazone; mitoxantrone

In particular embodiments of the invention, the chemotherapeutic agent is a topoisomerase inhibitor.

Any suitable topoisomerase inhibitor may be used in the present invention. In a particular embodiment, the topoisomerase inhibitor is a topoisomerase I 4 inhibitor, for example a camptothecin. A suitable topoisomerase I inhibitor, which may be used in the present invention is irenotecan (CPT-11) or its active metabolite SN-38. CPT-11 specifically acts in the S phase of the cell cycle by stabilizing a reversible covalent reaction intermediate, referred to as a cleavage or cleavage complex and may also induces G₂-M cell cycle arrest.

In certain embodiments of the invention, the chemotherapeutic agent is a fluoropyrimidine e.g. 5-FU.

Where reference is made to specific chemotherapeutic agents, it should be understood that analogues including biologically active derivatives and substantial equivalents thereof, which retain the antitumour activity of the specific agents, may be used.

### EGF Inhibitors

As described above, the inventors have found that combinations of two or more inhibitors of EGFS may be used to obtain a dramatically enhanced tumour cell growth attenuating effect. In certain embodiments of the invention, any molecule which reduces expression of an EGF gene or antagonizes the EGF protein may be used as the EGF inhibitor. In particular embodiments, the EGF is HB-EGF, AREG, TGF, EREG, BTC, or NRG3.

In one embodiment, inhibitors of HB-EGF and of AREG are used.

EGF inhibitors may include, but are not limited to, antibodies, antibody fragments, immunoconjugates, small molecule inhibitors, peptide inhibitors, specific binding members, non-peptide small organic molecules, antisense molecules, aptamers, or oligonucleotide decoys.

Any Erb1 or EGF receptor inhibitor should indirectly inhibit AREG activity. Suitable inhibitors include, but are not limited to, PD169540 (a pan-ErbB inhibitor) and IRESSA (an ErbBl-specific inhibitor).

Other suitable inhibitors may include CTyrphostin AG 1478 (a selective and potent inhibitor of EGF-R kinase) which indirectly inhibits TGF-alpha; ZM 252868 is an Epidermal growth factor (EGF) receptor-specific tyrosine kinase inhibitor which inhibits TGF-alpha actions in ovarian cancer cells (Simpson et al, British Journal of Cancer, 79(7-8):1098-103, 1999).

A suitable inhibitor of HB-EGF may include CRM197.

In one embodiment, an indirect inhibitor of the EGF-receptor may be utilised.

In another embodiment, the inhibitor a direct inhibitor of the EGF is used. In particular, embodiments, a direct inhibitor is an antibody molecule which binds EGF or a nucleic acid molecule which inhibits expression of said EGF.

In one embodiment, the inhibitor is an anti EGF antibody.

The inventors have developed some novel antibodies for use in the present invention. In a particular, embodiment, an antibody of or for use in the invention is an antibody molecule having binding specificity for AREG, wherein the antibody molecule is the 6E11 1E9 106 antibody, or a fragment thereof.

Antibody molecules of or for use in the invention herein include antibody fragments and “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences, in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An antibody molecule for use in the invention may be a bispecific antibody or bispecific fragment. For example, the antibody molecule or fragment may have specificity for HB-EGF and for AREG. For example, In one embodiment, a bispecific antibody molecule for use in the present invention may comprise a first heavy chain and a first light chain from the anti 6E11 1E9 106 and an additional antibody heavy chain and light chain with binding specificity for HB-EGF. A number of methods are known in the art for the production of antibody bispecific antibodies and fragments. For example, such methods include the fusion of hybridomas or linking of Fab′ fragments (for example, see Songsivilai & Lachmann, Clin. Exp. Immunol. 79: 315-321 (1990), Kostelny et al., J. Immunol. 148:1547-1553 (1992)). In another embodiement, bispecific antibodies may be formed as “diabodies”.

Antibody molecules, such as antibodies and antibody fragments, for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352: 624-628 and Marks et al. (1992) Bio/Technology, 10: 779-783). Other antibody production techniques are described in Using Antibodies: A Laboratory Manual, eds. Harlow and Lane, Cold Spring Harbor Laboratory, 1999.

Traditional hybridoma techniques typically involve the immunisation of a mouse or other animal with an antigen in order to elicit production of lymphocytes capable of binding the antigen. The lymphocytes are isolated and fused with a myeloma cell line to form hybridoma cells which are then cultured in conditions which inhibit the growth of the parental myeloma cells but allow growth of the antibody producing cells. The hybridoma may be subject to genetic mutation, which may or may not alter the binding specificity of antibodies produced. Synthetic antibodies can be made using techniques known in the art (see, for example, Knappik et al, J. Mol. Biol. (2000) 296, 57-86 and Krebs et al, J. Immunol. Meth. (2001) 215467-84.

Modifications may be made in the VH, VL or CDRs of the binding members, or indeed in the FRs using any suitable technique known in the art. For example, variable VH and/or VL domains may be produced by introducing a CDR, e.g. CDR3 into a VH or VL domain lacking such a CDR. Marks et al. (1992) Bio/Technology, 10: 779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences of the present invention may be produced.

Accordingly, antibodies and antibody fragments for use in the invention may be produced by a method comprising: (a) providing a starting repertoire of nucleic acids encoding a variable domain, wherein the variable domain includes a CDR1, CDR2 or CDR3 to be replaced or the nucleic acid lacks an encoding region for such a CDR; (b) combining the repertoire with a donor nucleic acid encoding an amino acid sequence such that the donor nucleic acid is inserted into the CDR region in the repertoire so as to provide a product repertoire of nucleic acids encoding a variable domain; (c) expressing the nucleic acids of the product repertoire; (d) selecting a specific antigen-binding fragment specific for said target; and (e) recovering the specific antigen-binding fragment or nucleic acid encoding it. The method may include an optional step of testing the specific binding member for ability to inhibit the activity of said target.

Alternative techniques of producing antibodies for use in the invention may involve random mutagenesis of gene(s) encoding the VH or VL domain using, for example, error prone PCR (see Gram et al, 1992, P.N.A.S. 893576-3580 Additionally or alternatively, CDRs may be targeted for mutagenesis e.g. using the molecular evolution approaches described by Barbas et al 1991 PNAS 3809-3813 and Scier 1996 J Mol Biol 263551-567.

An antibody for use in the invention may be a “naked” antibody (or fragment thereof) i.e. an antibody (or fragment thereof) which is not conjugated with an “active therapeutic agent”. An “active therapeutic agent” is a molecule or atom which is conjugated to a antibody moiety (including antibody fragments, CDRs etc) to produce a conjugate. Examples of such “active therapeutic agents” include drugs, toxins, radioisotopes, immunomodulators, chelators, boron compounds, dyes etc.

An EGF inhibitor for use in the invention may be in the form of an immunoconjugate, comprising an antibody fragment conjugated to an “active therapeutic agent”. The therapeutic agent may be a chemotherapeutic agent or another molecule.

Methods of producing immunoconjugates are well known in the art; for example, see U.S. Pat. No. 5,057,313, Shih et al., Int. J. Cancer 41: 832-839 (1988); Shih et al., Int. J. Cancer 46: 1101-1106 (1990), Wong, Chemistry Of Protein Conjugation And Cross-Linking (CRC Press 1991); Upeslacis et al., “Modification of Antibodies by Chemical Methods,” in Monoclonal Antibodies: Principles And Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering And Clinical Application, Ritter et al.(eds.), pages 60-84 (Cambridge University Press 1995).

The antibody molecules for use in the invention may comprise further modifications. For example the antibody molecules can be glycosylated, pegylated, or linked to albumin or a nonproteinaceous polymer.

### Antisense/siRNA

Inhibitors of EGF and inhibitors of HB-EGF for use in the present invention may comprise nucleic acid molecules capable of modulating gene expression, for example capable of down regulating expression of a sequence encoding an EGF protein. Such nucleic acid molecules may include, but are not limited to antisense molecules, short interfering nucleic acid (siNA), for example short interfering RNA (siRNA), double-stranded RNA (dsRNA), micro RNA, short hairpin RNA (shRNA), nucleic acid sensor molecules, allozymes, enzymatic nucleic acid molecules, and triplex oligonucleotides and any other nucleic acid molecule which can be used in mediating RNA interference “RNAi” or gene silencing in a sequence-specific manner (see for example Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914; Allshire, 2002, Science, 297, 1818-1819; Volpe et al., 2002, Science, 297, 1833-1837; Jenuwein, 2002, Science, 297, 2215-2218; Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831).

An “antisense nucleic acid”, is a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). The antisense molecule may be complementary to a target sequence along a single contiguous sequence of the antisense molecule or may be in certain embodiments, bind to a substrate such that the substrate, the antisense molecule or both can bind such that the antisense molecule forms a loop such that the antisense molecule can be complementary to two or more non-contiguous substrate sequences or two or more non-contiguous sequence portions of an antisense molecule can be complementary to a target sequence, or both. Details of antisense methodology are known in the art, for example see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

A “triplex nucleic acid” or “triplex oligonucleotide” is a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504).

### Aptamers

Aptamers are nucleic acid (DNA and RNA) macromolecules that bind tightly to a specific moledular target. They can be produced rapidly through repeated rounds of in vitro selection for example by SELEX (systematic evolution of ligands by exponential enrichment) to bind to various molecular targets such as small molecules, proteins, nucleic acids etc (see Ellington and Szostak, Nature 346(6287):818-822 (1990), Tuerk and Gold, Science 249(4968):505-510 (1990) U.S. Pat. No. 6,867,289; U.S. Pat. No. 5,567,588, U.S. Pat. No. 6,699,843).

In addition to exhibiting remarkable specificity, aptamers generally bind their targets with very high affinity; the majority of anti-protein aptamers have equilibrium dissociation constants (Kds) in the picomolar (pM) to low nanomolar (nM) range. Aptamers are readily produced by chemical synthesis, possess desirable storage properties, and elicit little or no immunogenicity in therapeutic applications.

Non-modified aptamers are cleared rapidly from the bloodstream, with a half-life of minutes to hours, mainly due to nuclease degradation and renal clearance a result of the aptamer's inherently low molecular weight. However, as is known in the art, modifications, such as 2′-fluorine-substituted pyrimidines, polyethylene glycol (PEG) linkage, etc. (can be used to adjust the half-life of the molecules to days or weeks as required.

Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein which has good solubility and compacity properties. Aptamers may comprise any deoxyribonucleotide or ribonucleotide or modifications of these bases, such as deoxythiophosphosphate (or phosphorothioate), which have sulfur in place of oxygen as one of the non-bridging ligands bound to the phosphorus. Monothiophosphates αS have one sulfur atom and are thus chiral around the phosphorus center. Dithiophosphates are substituted at both oxygens and are thus achiral. Phosphorothioate nucleotides are commercially available or can be synthesized by several different methods known in the art.).

### Treatment

“Treatment” or “therapy” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and/or vascularisation and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, prostate, cervical and ovarian carcinoma, non-small cell lung cancer, hepatocellular carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, astrocytomas, gliomas and retinoblastomas.

The invention may be particularly useful in the treatment of existing cancer and in the prevention of the recurrence of cancer after initial treatment or surgery.

### Pharmaceutical Compositions

Pharmaceutical compositions according to the present invention, and for use in accordance with the present invention may comprise, in addition to active ingredients, e.g (i) a chemotherapeutic agent and/or an EGF inhibitor or (ii) an inhibitor of a first EGF and an inhibitor of a second EGF, a pharmaceutically acceptable excipient, a carrier, buffer stabiliser or other materials well known to those skilled in the art (see, for example, (Remington: the Science and Practice of Pharmacy, 21^{st} edition, Gennaro A R, et al, eds., Lippincott Williams & Wilkins, 2005.). Such materials may include buffers such as acetate, Tris, phosphate, citrate, and other organic acids; antioxidants; preservatives; proteins, such as serum albumin, gelatin, or immunoglobulins; hydrophilic polymers such aspolyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compound selected as necessary for the particular indication being treated, preferably with complementary activities that do not adversely affect the activity of the composition of the invention. For example, in the treatment of cancer, in addition to one or more EGF inhibitors and/or a chemotherapeutic agent, the formulation or kit may comprise an additional component, for example a second or further EGF inhibitor, a second or further chemotherapeutic agent, or an antibody to a target other than the EGF to which the said inhibitors bind, for example to a growth factor which affects the growth of a particular cancer.

The active ingredients (e.g. EGF inhibitors, for example HB-EGF inhibitors, AREG inhibitors, and/or chemotherapeutic agents) may be administered via microspheres, microcapsules liposomes, other microparticulate delivery systems. For example, active ingredients may be entrapped within microcapsules which may be prepared, for example, by coacervation techniques or by interfacial polymerization, for example, hydroxymethylcellulose or gelatinmicrocapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 21^{st} edition, Gennaro A R, et al, eds., Lippincott Williams & Wilkins, 2005.

Sustained-release preparations may be used for delivery of active agents. Suitable examples of sustained-release preparations include semi-permeable matrices of solid hydrophobic polymers containing the antibody, which matrices are in the form of shaped articles, e.g. films, suppositories or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid andy ethyl-Lglutamate, non-degradable ethylene-vinyl acetate, degradable lactic acid-glycolic acid copolymers, and poly-D-(−)-3-hydroxybutyric acid.

As described above nucleic acids may also be used in methods of treatment. Nucleic acid for use in the invention may be delivered to cells of interest using any suitable technique known in the art. Nucleic acid (optionally contained in a vector) may be delivered to a patient's cells using in vivo or ex vivo techniques. For in vivo techniques, transfection with viral vectors (such as adenovirus, Herpes simplex I virus, or adeno-associated virus) and lipid-based systems (useful lipids for lipid-mediated transfer of the gene are DOTMA, DOPE and DC-Chol, for example) may be used (see for example, Anderson et al., Science 256: 808-813 (1992). See also WO 93/25673).

In ex vivo techniques, the nucleic acid is introduced into isolated cells of the patient with the modified cells being administered to the patient either directly or, for example, encapsulated within porous membranes which are implanted into the patient (see, e.g. U.S. Pat. Nos. 4,892,538 and 5,283,187). Techniques available for introducing nucleic acids into viable cells may include the use of retroviral vectors, liposomes, electroporation, microinjection, cell fusion, DEAE-dextran, the calcium phosphate precipitation method, etc.

The EGF inhibitors and/or chemotherapeutic agent may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

In embodiments of the invention, where an EGF inhibitor and a chemotherapeutic agent are used in treatment, the EGF inhibitor may be administered simultaneously, separately or sequentially with the chemotherapeutic agent. Likewise, in embodiments of the invention, where a first EGF inhibitor and a second EGF inhibitor are used in treatment together, the first EGF inhibitor may be administered simultaneously, separately or sequentially with the second EGF inhibitor. Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

### Kits

The invention further extends to various kits for the treatment of cancer or the killing of tumour cells. The kits may optionally include instructions fok the administration of each component, e.g. EGF inhibitor and chemotherapeutic agent, or first EGF inhibitor and second EGF inhibitor, separately, sequentially or simultaneously.

### Dose

The EGF inhibitors and/or chemotherapeutic agents of and for use in the invention are suitably administered to an individual in a “therapeutically effective amount”, this being sufficient to show benefit to the individual. The actual dosage regimen will depend on a number of factors including the condition being treated, its severity, the patient being treated, the agents being used, and will be at the discretion of the physician.

In one embodiment of the methods, compositions or kits, in which an EGF inhibitor and a chemotherapeutic agent is used, the EGF inhibitor and chemotherapeutic agent are administered in doses which produce a potentiating ratio. Likewise, in one embodiment of the methods, compositions or kits, in which a first EGF inhibitor and a second EGF inhibitor is used, the EGF inhibitor and chemotherapeutic agent are administered in doses which produce a potentiating ratio.

The term “potentiating ratio” in the context of the present invention is used to indicate that two components, e.g. EGF inhibitors, chemotherapeutic agents etc. are present in a ratio such that the cytotoxic activity of the combination is greater than that of either component alone or of the additive activity that would be predicted for the combinations based on the activities of the individual components.

Thus in a potentiating ratio, the individual components act synergistically.

Synergism may be defined using a number of methods.

In one method, synergism may be determined by calculating the combination index (CI) according to the method of Chou and Talalay. CI values of 1, <1, and >1 indicate additive, synergistic and antagonistic effects respectively.

In one embodiment of the invention, the EGF inhibitor and the chemotherapeutic agent are present in concentrations sufficient to produce a CI of less than 1, such as less than 0.85. Likewise, in another embodiment of the invention, the first EGF inhibitor and the second EGF inhibitor are present in concentrations sufficient to produce a CI of less than 1, such as less than 0.85.

Synergism is preferably defined as an RI of greater than unity using the method of Kern as modified by Romaneli (1998a, b). The R1 may be calculated as the ratio of expected cell survival (Sep, defined as the product of the survival observed with component A alone and the survival observed with component B alone) to the observed cell survival (Sobs) for the combination of A and B(RI=Se/Sobs). Synergism may then be defined as an RI of greater than unity.

In one embodiment of the invention, the EGF inhibitor and the chemotherapeutic agent (or the first EGF inhibitor and the second EGF inhibitor) are provided in concentrations sufficient to produce an RI of greater than 1.5, such as greater than 2.0, for example greater than 2.25.

Thus in one embodiment the combined medicament produces a synergistic effect when used to treat tumour cells.

The optimal dose can be determined by physicians based on a number of parameters including, for example, age, sex, weight, severity of the condition being treated, the active ingredient being administered and the route of administration.

The invention will now be described further in the following non-limiting examples with reference made to the accompanying drawings in which:

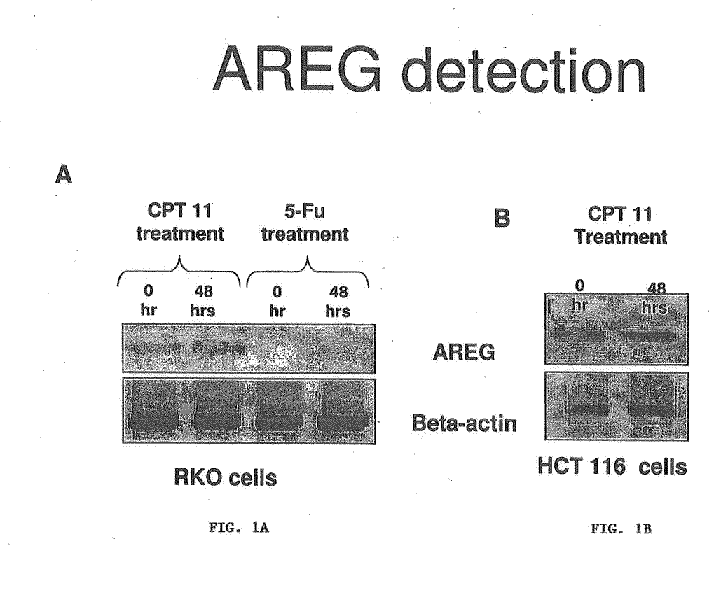

FIG. 1A illustrates analysis of AREG and beta actin RNA expression in RKO +/+ with/without either a 48 hour CPT11 treatment or 5-Fu treatment. RNA levels were analyzed following 35 cycle of PCR to determine relative differences in expression between treated and untreated samples;

FIG. 1B illustrates analysis of AREG and beta actin RNA expression in HCT116 +/+ with/without a 48 hour CPT11 treatment. RNA levels were analyzed following 35 cycles of PCR to determine relative differences in expression between treated and untreated samples;

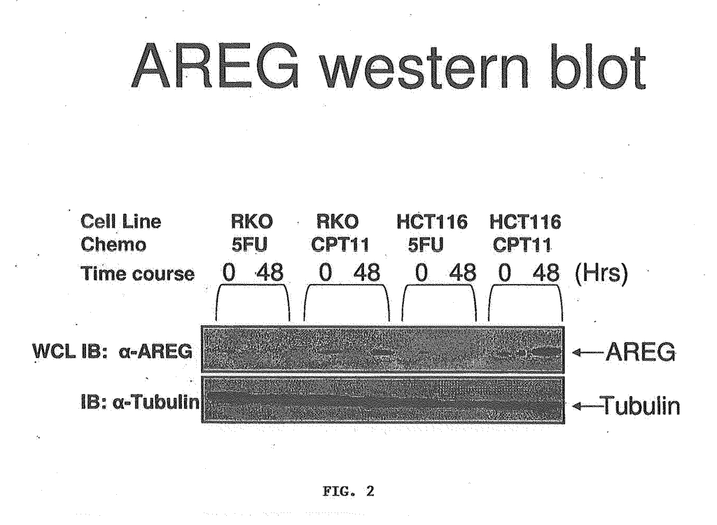

FIG. 2 illustrates western blot analysis of AREG and gamma tubulin protein expression in HCT116 +/+ and RKO +/+ with/without a 48 hour CPT11 or 5-Fu treatment;

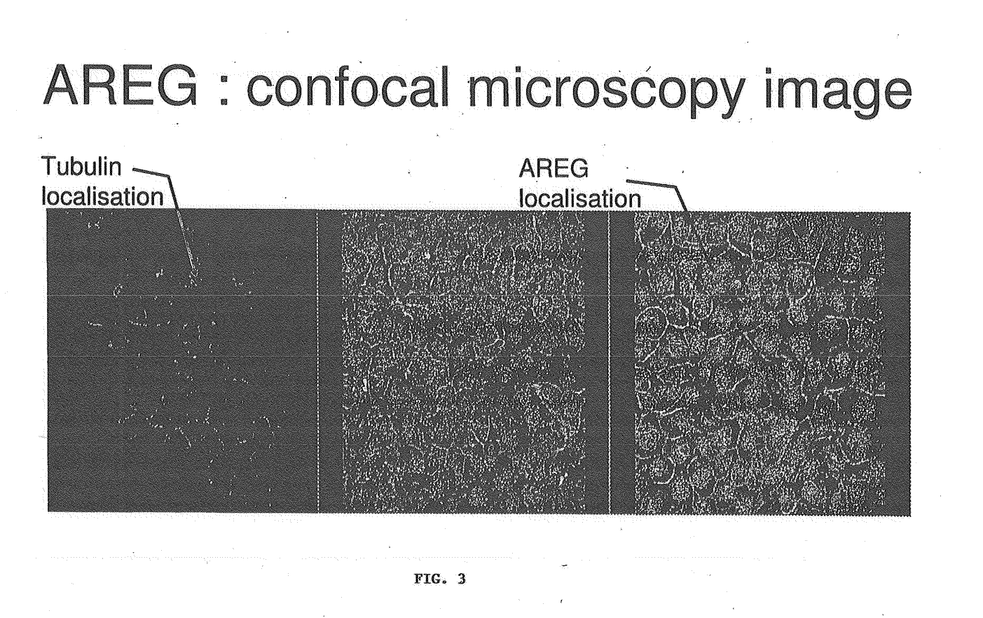

FIG. 3 illustrates confocal microscopy image of AREG and Actin protein in HCT 116 +/+ with or without CPT-11 treatment;

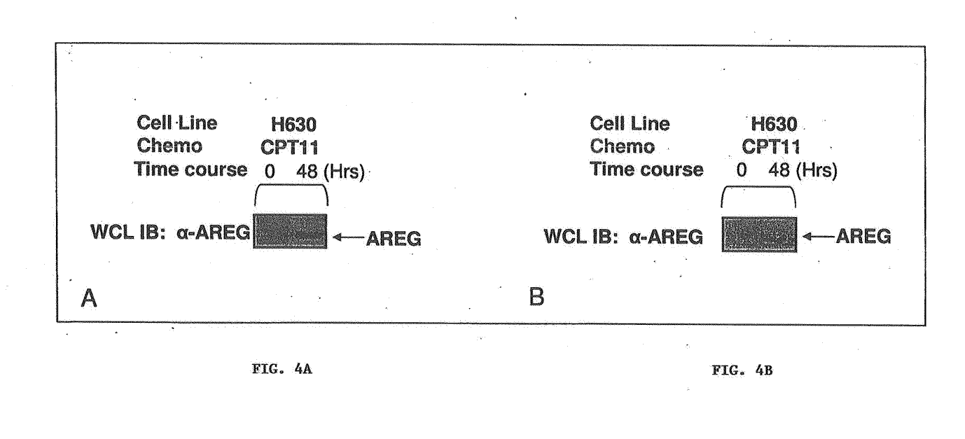

FIG. 4 illustrates analysis of AREG protein 14, expression in H630 p53 mutant colorectal cancer cell lysates following a 48 hour CPT11 treatment. Western blots were probed using an anti-amphiregulin antibody. Enhanced AREG expression was observed following chemotherapy (A and B illustrate two separate experiments).

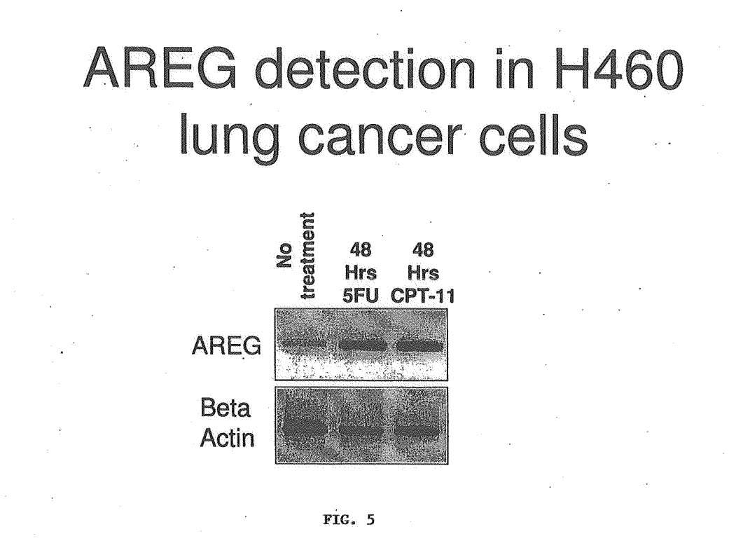

FIG. 5 illustrates analysis of AREG and beta actin RNA expression in H460 lung cancer cells with/without either a 48 hour CPT11 treatment or 5-Fu treatment. RNA levels were analyzed following 35 cycle of PCR to determine relative differences in expression between treated and untreated samples.

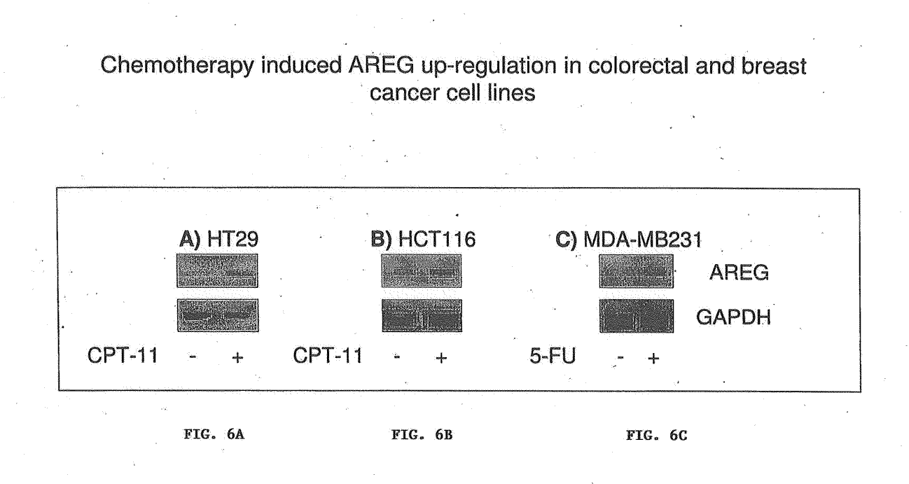

FIG. 6 illustrates AREG upregulation following chemotherapeutic challenge in A) HT29, B) HCT116 and C) MDA-MB231 cells. Cells were treated with/without chemotherapy for 48 hours. RT-PCR was performed with 1 μg of total RNA using primer pairs specific for the human AREG gene or GAPDH. The PCR products were separated on 1.5% agarose gel electrophoresis and visualized by ethidium bromide staining.

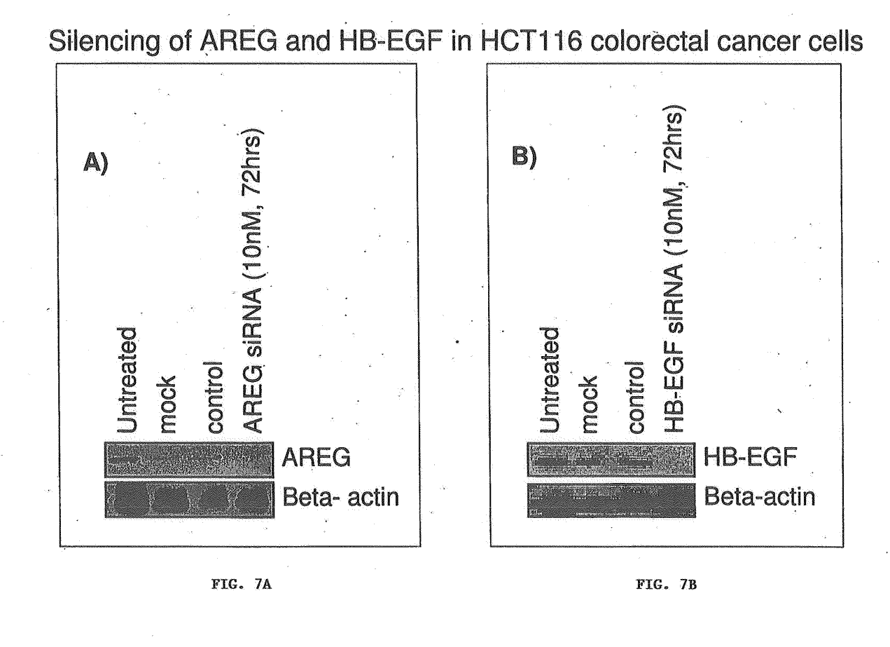

FIG. 7a illustrates specific AREG silencing by siRNA in HCT116. HCT116 cells were transfected with AREG specific siRNA (10 nM), or a control siRNA (10 nM). AREG and Beta-actin gene expression were measured by RT-PCR RNA 72 hrs after transfection. FIG. 7b) illustrates specific HB-EGF silencing by siRNA in HCT116. HCT116 cells were transfected with HB-EGF specific siRNA (10 nM), or a control siRNA (10 nM). HB-EGF and Beta-actin gene expression were measured by RT-PCR RNA 72 hrs after transfection

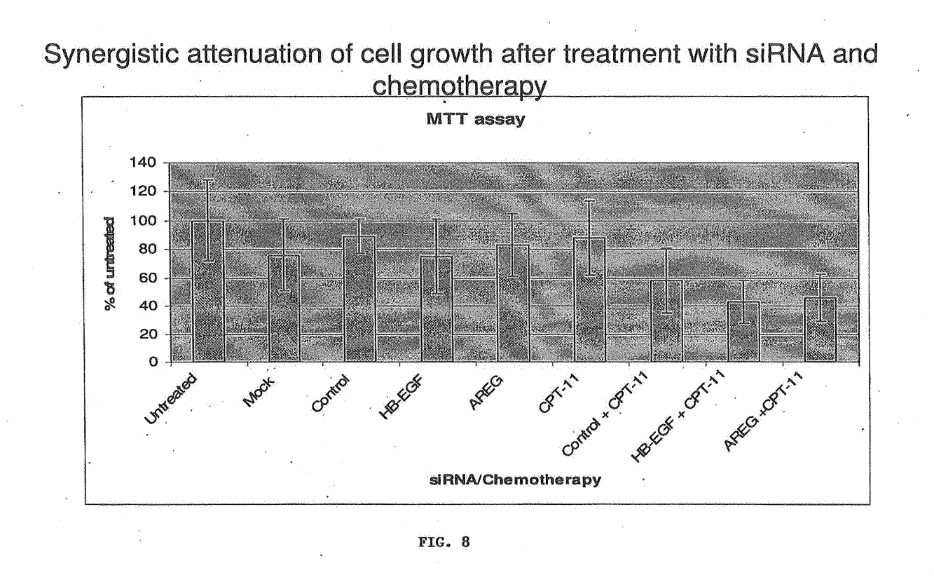

FIG. 8 illustrates HCT116 cell proliferation following AREG/HB-EGF silencing by siRNA and/or Chemotherapy treatment. Cells were transfected with AREG siRNA (10 nM), HB-EGF siRNA (10 nM) or a control siRNA (10 nM). Transfected cells were treated with no drug or 3.5 μM CPT-11. Cell proliferation was analysed by MTT assay 48 hr after transfection/chemotherapy

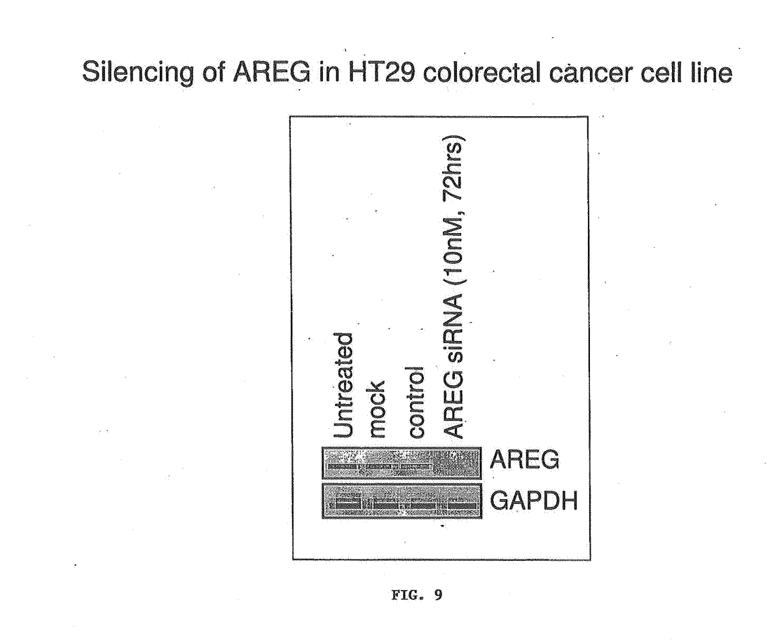

FIG. 9 illustrates specific AREG silencing by siRNA in HT29 colorectal cancer cells. Cells were transfected with AREG specific siRNA (10 nM), or a control siRNA (10 nM). AREG and GAPDH gene expression were measured by RT-PCR RNA 72 hrs after transfection.

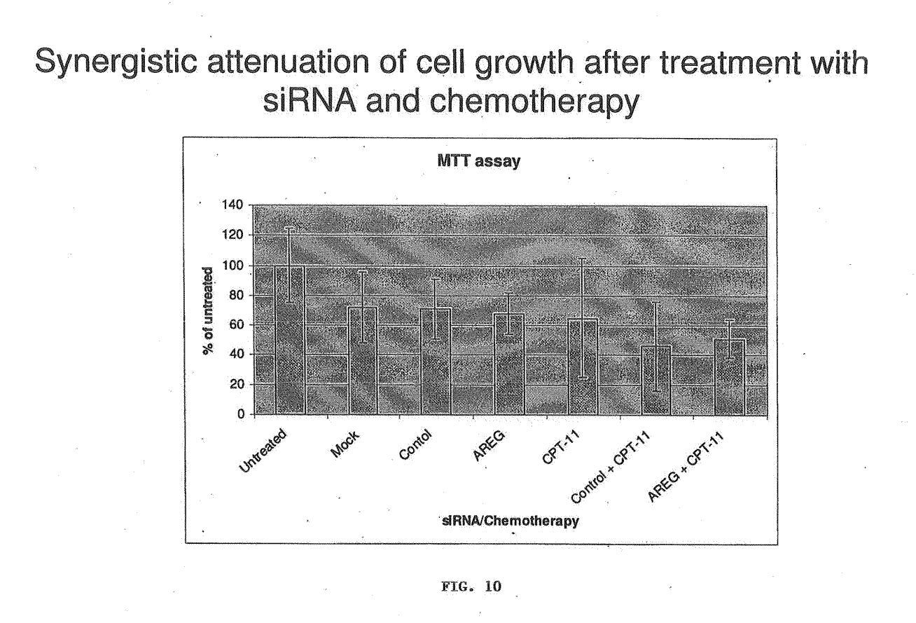

FIG. 10 illustrates HT29 cell proliferation following AREG silencing by siRNA and/or Chemotherapy treatment. Cells were transfected with AREG siRNA (10 nM) or a control siRNA (10 nM). Transfected cells were treated with no drug or 3.5 μM CPT-11. Cell proliferation was analysed by MTT assay 48 hr after transfection/chemotherapy.

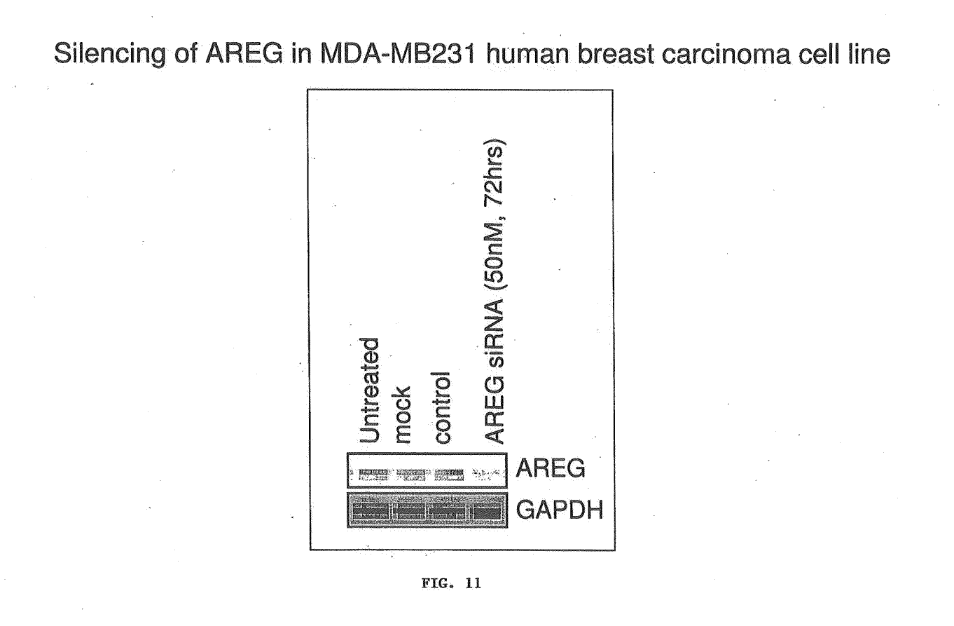

FIG. 11 illustrates Specific AREG silencing by siRNA in MDA-MB231 cells. Cells were transfected with AREG specific siRNA (10 nM), or a control siRNA (10 nM). AREG and GAPDH gene expression were measured by RT-PCR RNA 72 hrs after transfection.

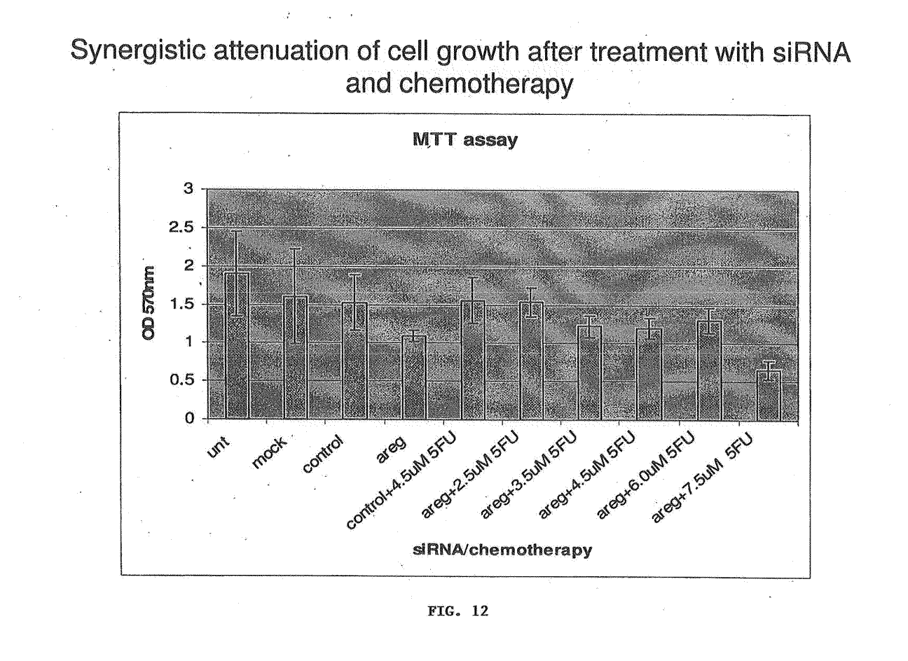

FIG. 12 illustrates MDA-MB231 cell proliferation following AREG silencing by siRNA and/or chemotherapy treatment. Cells were transfected with AREG siRNA (10 nM), or a control siRNA (10 nM) and varying doses of 5-FU. Cell proliferation was analysed by MTT assay 48 hr after transfection/chemotherapy.

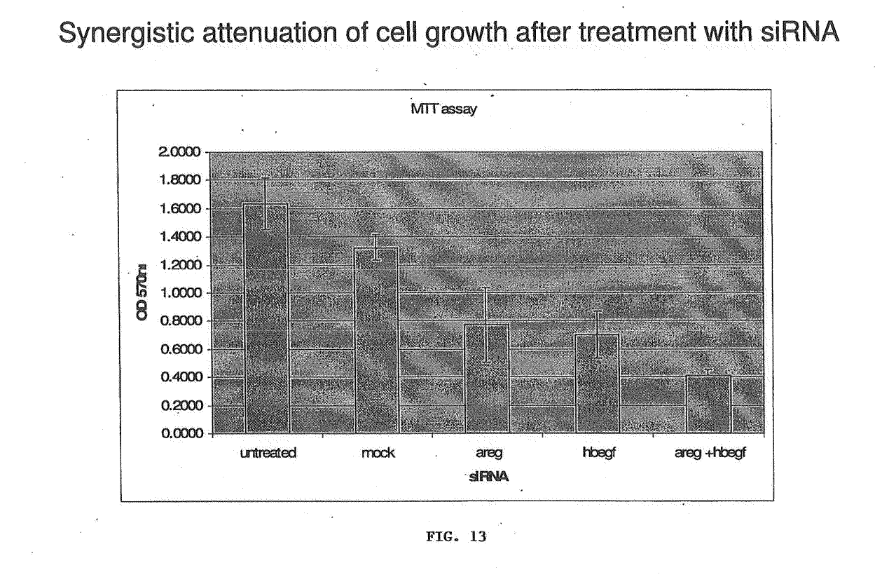

FIG. 13 illustrates MDA-MB231 cell proliferation following AREG/HB-EGF silencing by siRNA. Cells were transfected with AREG siRNA (10 nM), HB-EGF siRNA (10 nM) or a control siRNA (10 nM). Cell proliferation was analysed by MTT assay 48 hr after transfection/chemotherapy.

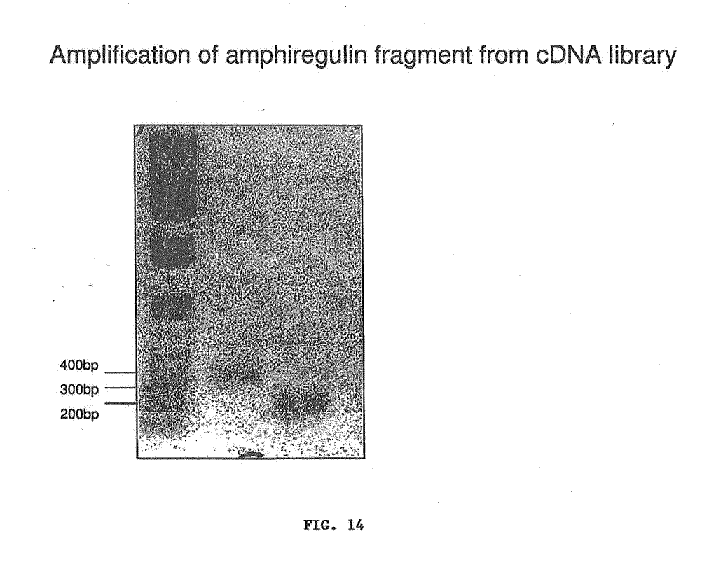

FIG. 14 illustrates amplification of amphiregulin fragments from cDNA library. Amphiregulin was amplified from kidney cDNA and PCR reaction was analysed on 1.5% agarose gel stained with ethidium bromide

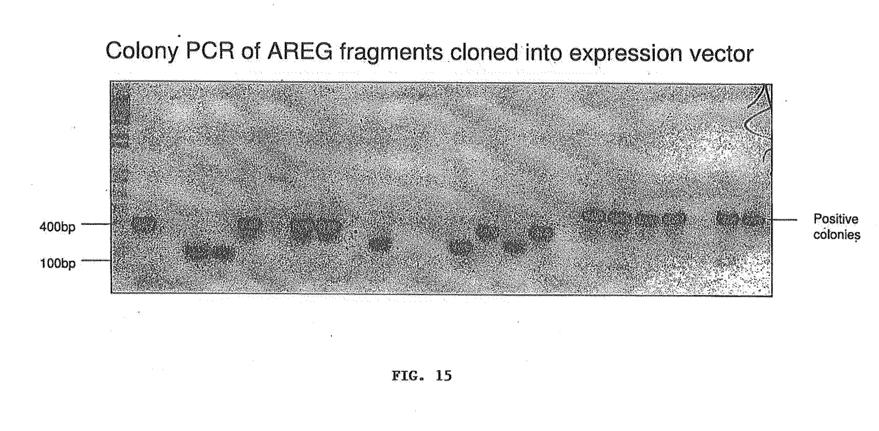

FIG. 15 illustrates colony PCR of AREG fragments. PCR amplification was carried out colonies to identify colonies that had the amphiregulin fragment successful cloned into the expression vector. PCR reaction was analysed on 1.5% agarose gel stained with ethidium bromide. Any positive colonies were selected for sequence and expression analysis

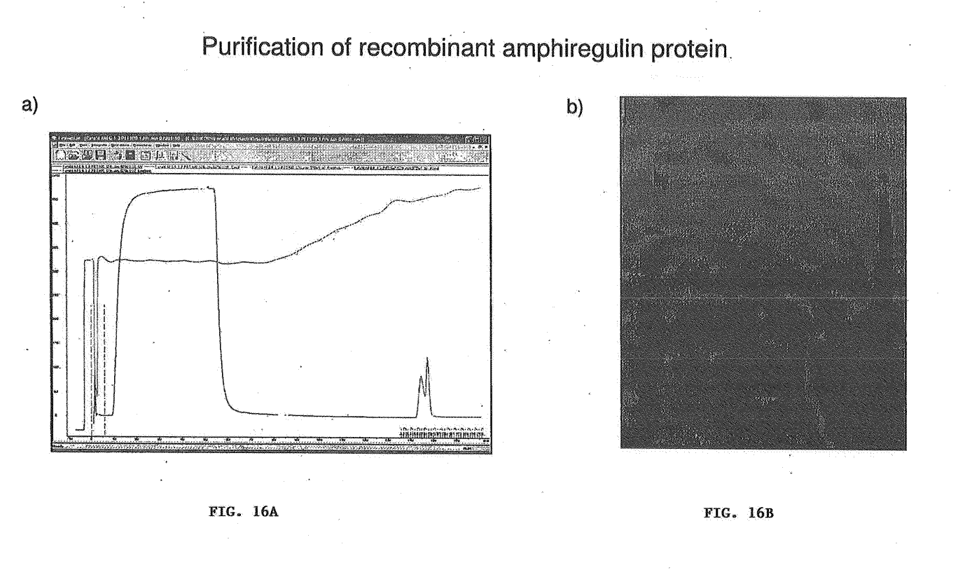

FIG. 16 Panel A) illustrates the elution profile of the purification of AREG recombinant protein from 500 ml culture volume. Pellet from culture was resuspended in 8M Urea and then purified by mature of the 6×Histidine tag. The elution samples were collected and analysed by SDS-PAGE (Panel B). The gel was stained with comassie blue.

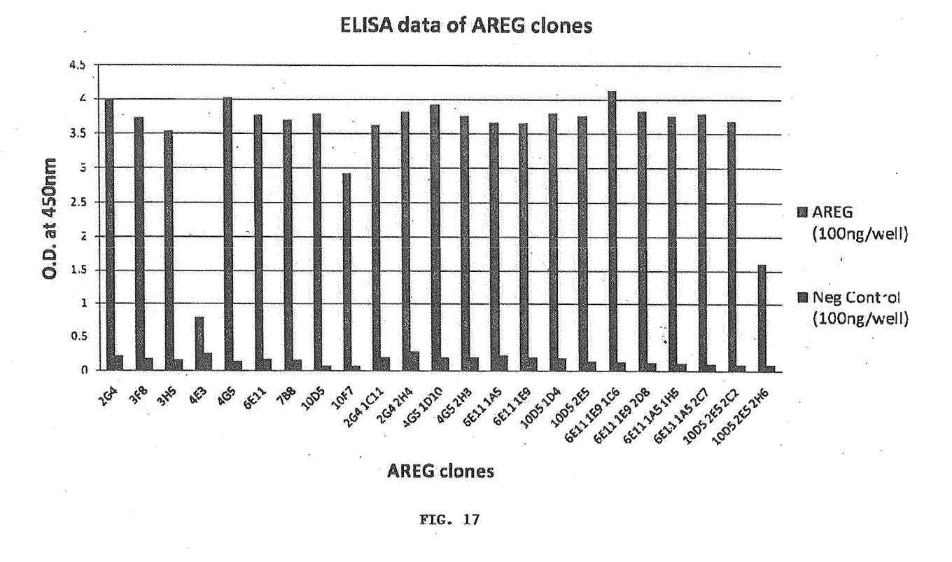

FIG. 17 illustrates ELISA result of AREG monoclonal antibodies produced. Monoclonal antibodies were tested by ELISA against recombinant AREG protein and a negative control produced in similar method.

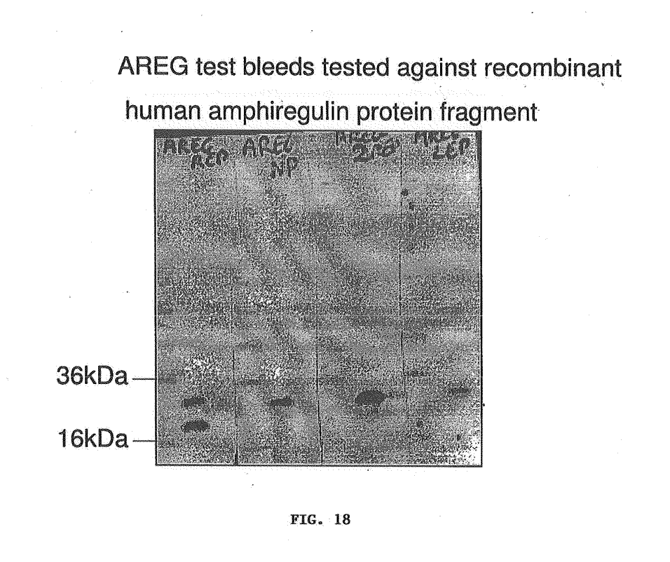

FIG. 18 illustrates western blot analysis of monoclonal test bleeds. The monoclonal test bleeds were tested by western blot against the recombinant amphiregulin protein and a negative control protein. Equal amounts of protein were loaded on SDS-PAGE gel.

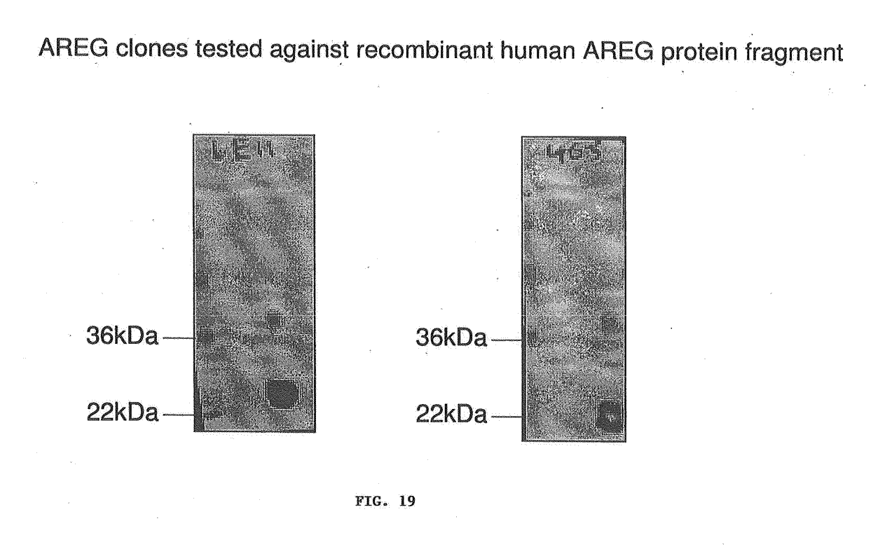

FIG. 19 illustrates western blot analysis of AREG monoclonal antibodies against recombinant protein. Recombinant AREG protein and a negative control protein were run on SDS-PAGE gel. The gel was transferred to nitrocellulose membrane and probed with the AREG monoclonal antibodies.

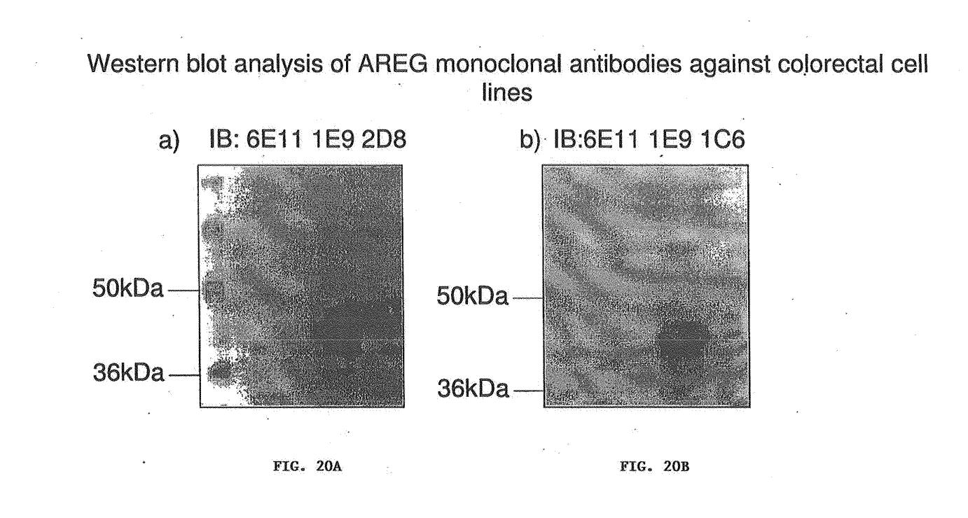

FIG. 20 illustrates western blot analysis of AREG monoclonal antibodies against whole cell lysated from colorectal cell lines HCT116 and HT29. Whole cell lysates from HCT116 and HT29 cell lines were prepared and ran on SDS-PAGE. Blots were probed with AREG monoclonal antibodies a) 6E11 1E92D8 and b) 6E11 1E9 106. (NB 6E11 1E92D8 has been subsequently shown to be the same antibody as 6E11 1E9 106).

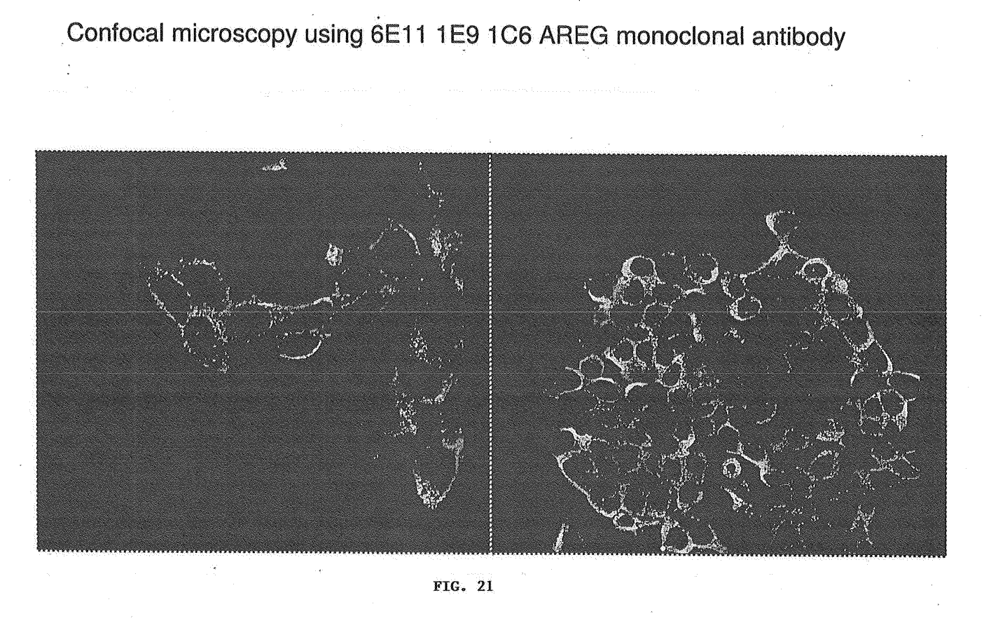

FIG. 21 illustrates confocal microscopy image of AREG and actin protein in HCT 116 +/+ stained with 6E11 1E9 106 Monoclonal antibody

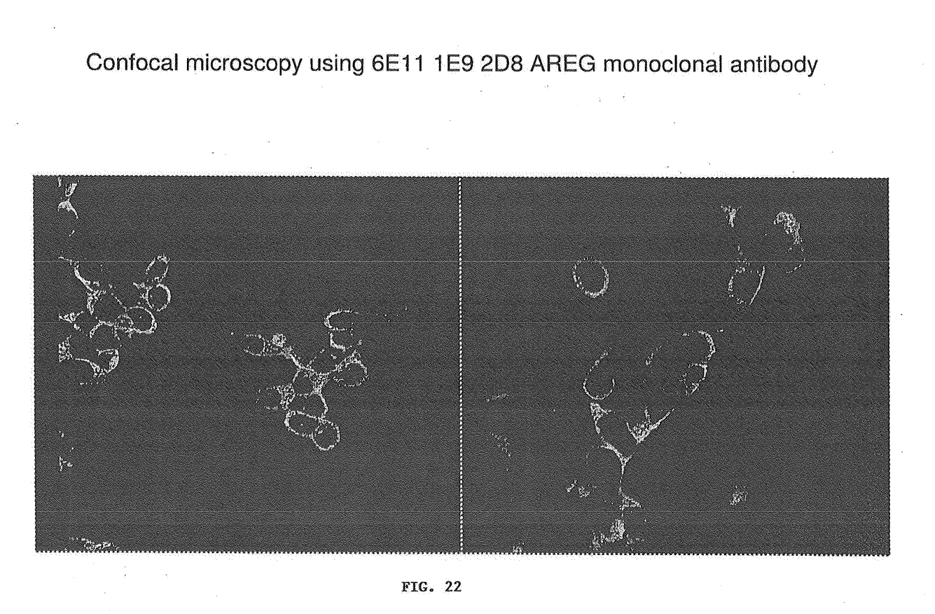

FIG. 22 illustrates confocal microscopy image of AREG and actin protein in HCT 116 +/+ stained with 6E11 1E92D8 Monoclonal antibody

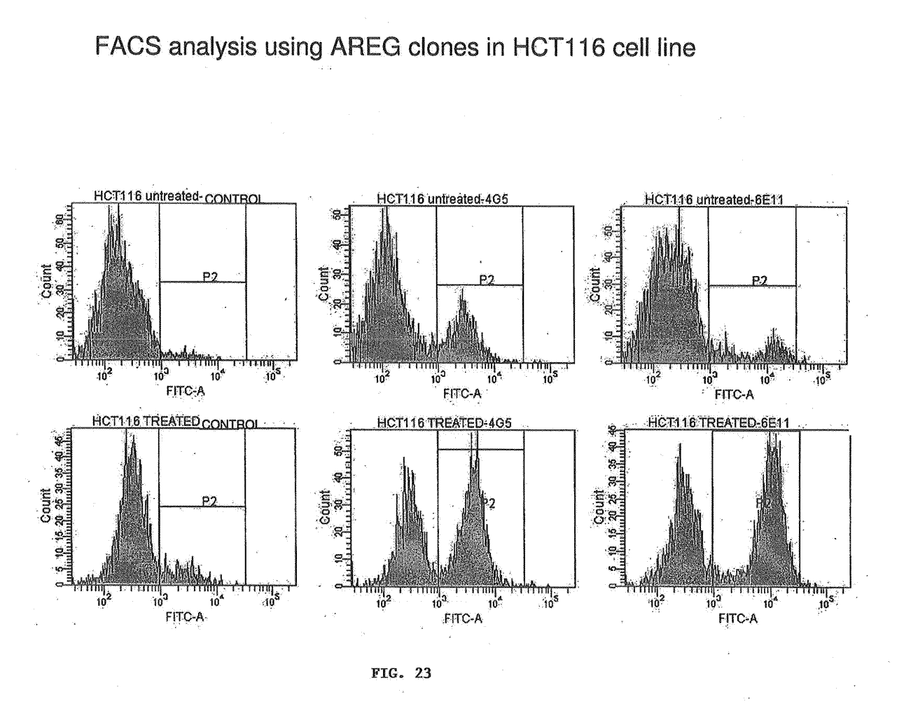

FIG. 23 illustrates FACS analysis in HCT116 colorectal cancer cell line treated with or without 2.5 μM irinotecan for 48 hours.

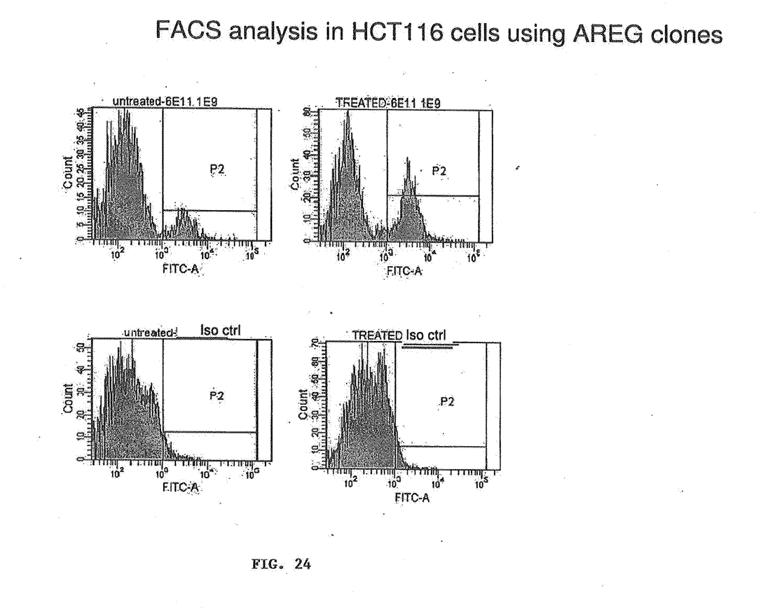

FIG. 24 illustrates FACS analysis in HCT116 cells treated with or without 2.5 μM irinotecan for 48 hours. Following treatment cells were stained with AREG monoclonal antibodies and analysed by FACS

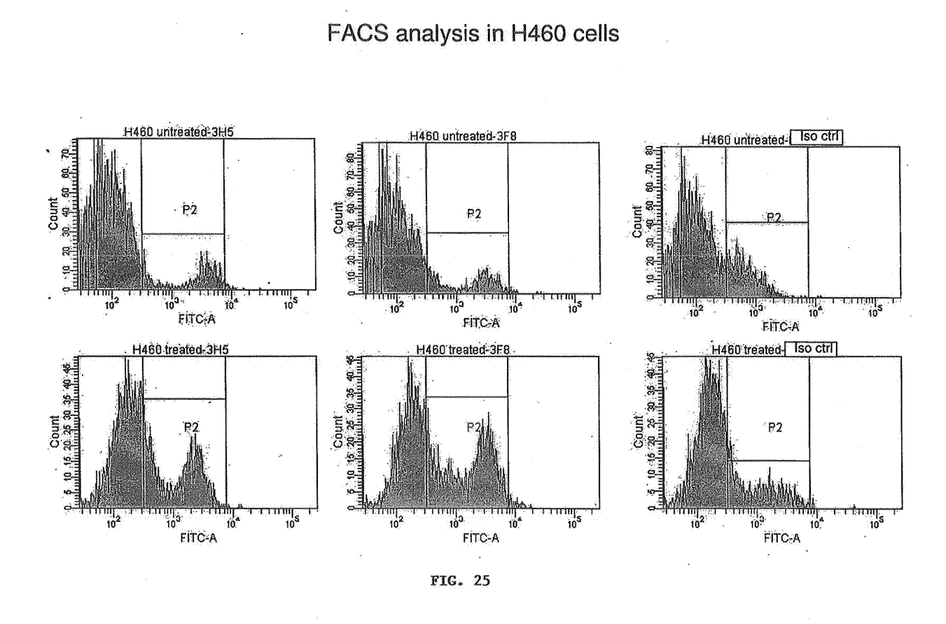

FIG. 25 illustrates FACS analysis in H460 lung carcinoma cell line treated with or without 2.5 μM irinotecan for 48 hours. Following treatment cells were stained with AREG monoclonal antibodies and analysed by FACS

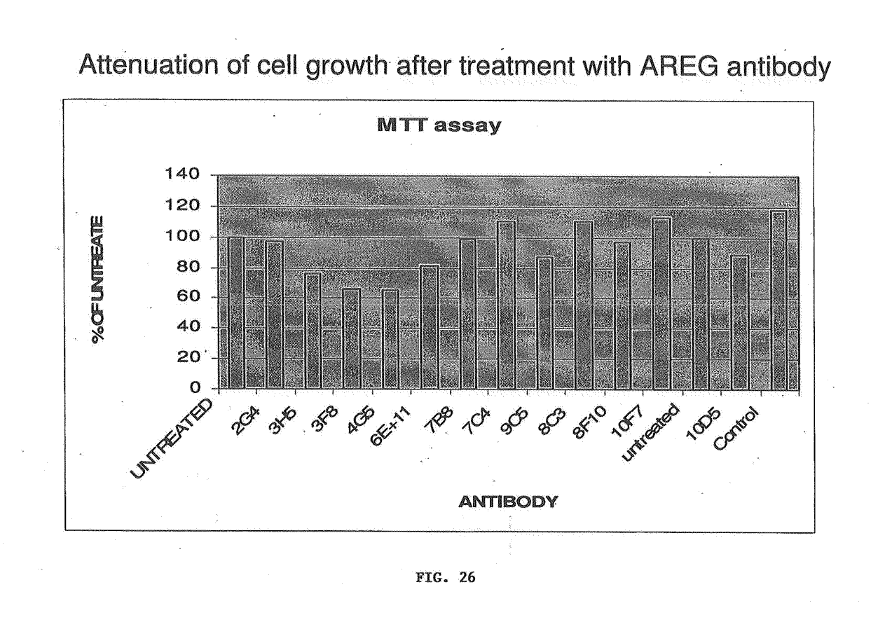

FIG. 26 illustrates MDA MB231 cell proliferation after treatment with AREG antibody. Cell proliferation was analysed by MTT assay 48 hr after treatment

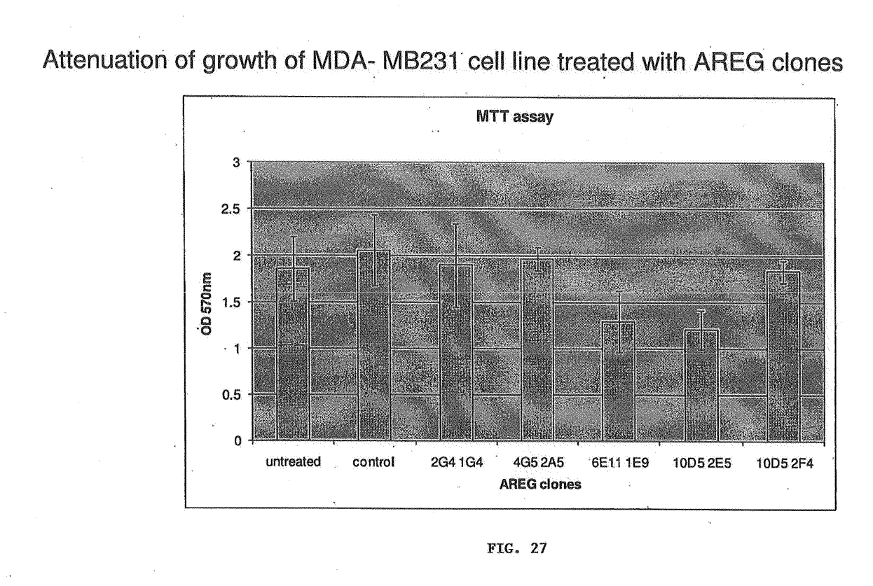

FIG. 27 illustrates MDA MB231 cell proliferation after treatment with AREG antibody: Cell proliferation was analysed by MTT assay 48 hr after treatment.

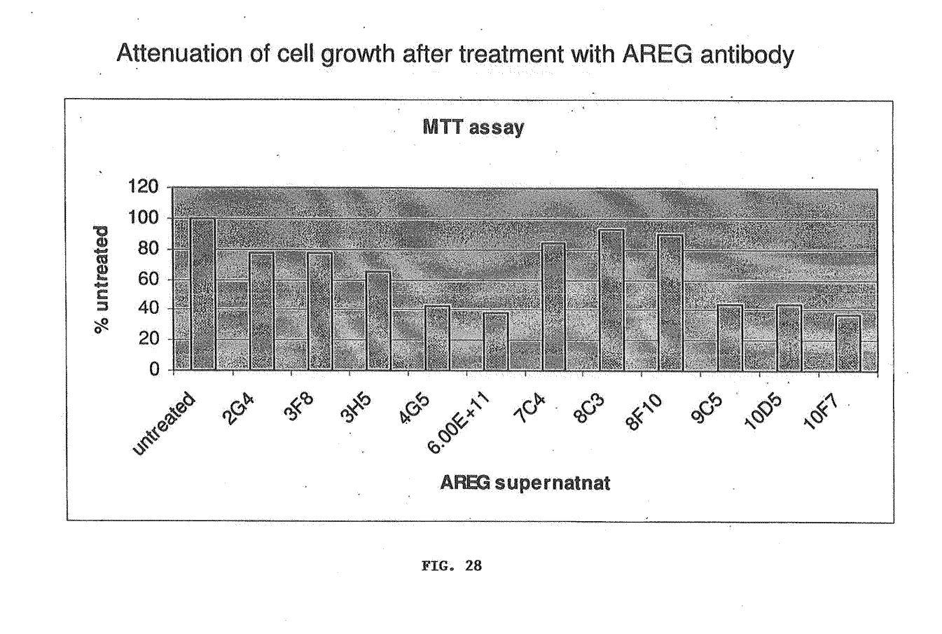

FIG. 28 illustrates HCT116 cell proliferation after treatment with AREG antibody. Cell proliferation was analysed by MTT assay 48 hr after treatment.

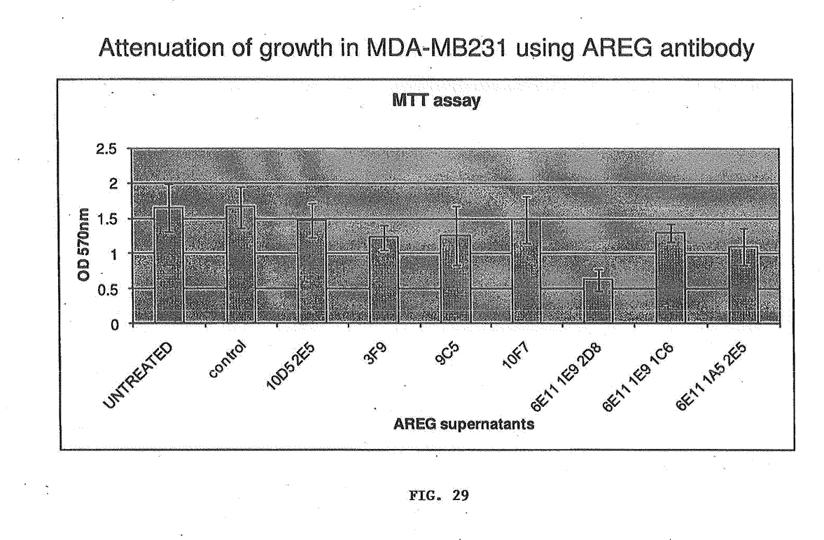

FIG. 29 illustrates MDA MB231 cell proliferation after treatment, with AREG antibody. Cell proliferation was analysed by MTT assay 48 hr after treatment.

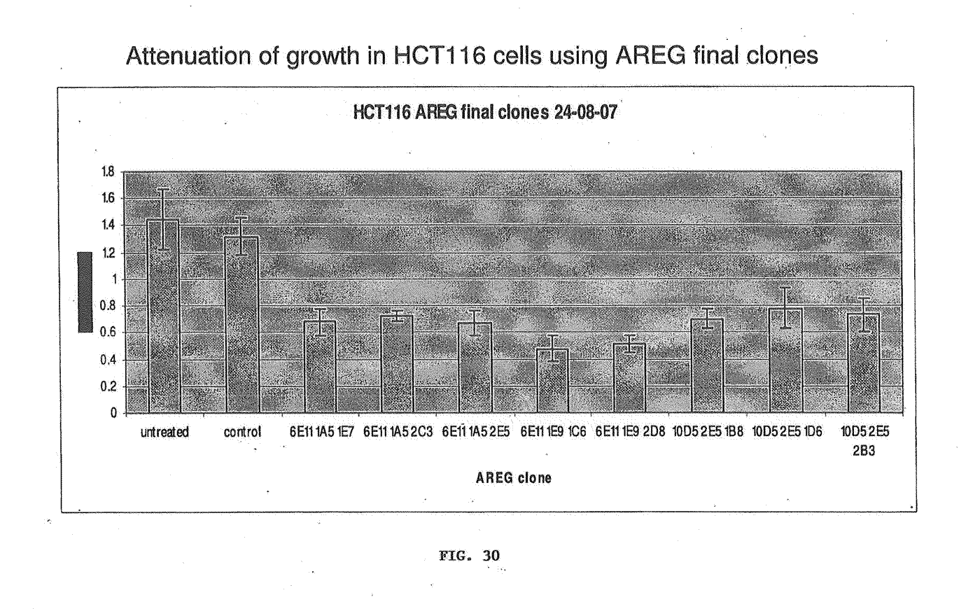

FIG. 30 illustrates HCT116 cell proliferation after treatment with AREG antibody. Cell proliferation was analysed by MTT assay 48 hr after treatment.

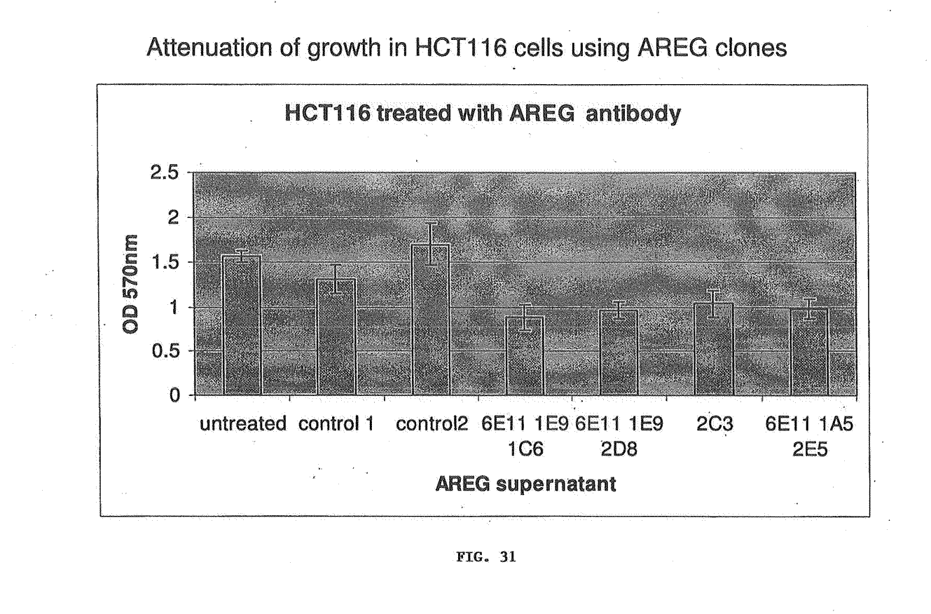

FIG. 31 illustrates HCT116 cell proliferation after treatment with AREG antibody. Cell proliferation was analysed by MTT assay 48 hr after treatment

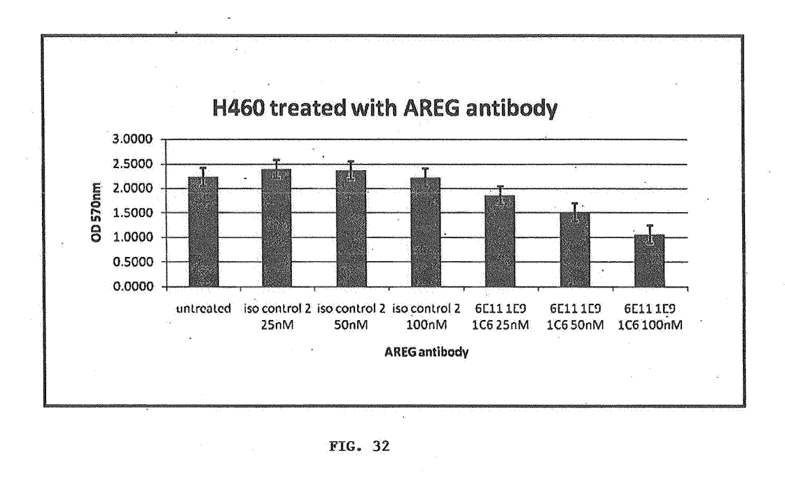

FIG. 32 illustrates H460 lung carcinoma cell proliferation following treatment with different concentrations of AREG (6E11 1E9 106) antibody or an isotype control antibody. Cells were seeded 24 hours before treatment with either 25 nM, 50 nM or 100 nM antibody. Cell viability was assayed 48 hours after treatment by MTT assay.

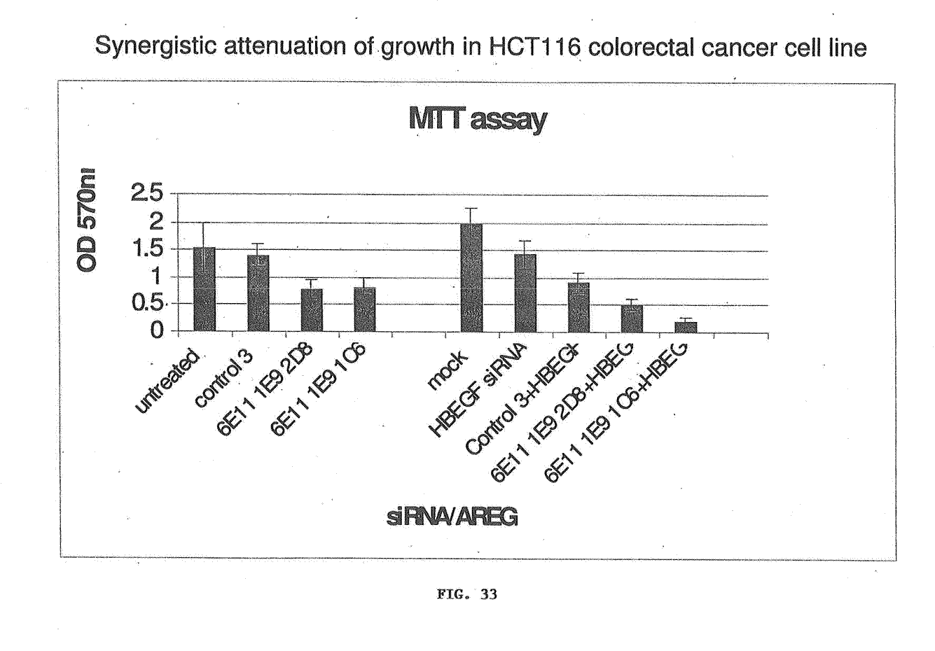

FIG. 33 illustrates HCT116 cell proliferation following HB-EGF silencing by siRNA and/or treatment with Anti AREG antibodies (6E11 1E92D8 & 6E11 1E9 106). Cells were transfected with HB-EGF siRNA (50 nM) or a control siRNA (50 nM). Cell proliferation was analysed by MTT assay 72 hr after transfection.

### MATERIALS AND METHODS

### Cell Lines and Culture Conditions

The HCT116 (p53 wild type) human colorectal adenocarcinoma cell line was maintained in McCoys (Invitrogen, UK). The RKO (p53 wild type) colorectal carcinoma cell line, the MDA-MB231 human breast carcinoma cell line and the HT29 human colorectal carcinoma cell line were each maintained in Dulbecco's Modified Eagle's Medium (DMEM, Invitrogen, UK).

colorectal cell lines were maintained in Dulbecco's Modified Eagle's Medium (DMEM, Invitrogen, UK). The HH630 (p53 mutant) colorectal cancer cell lines were maintained in Dulbecco's Modified Eagle's Medium (DMEM, Invitrogen, UK). The H460 (p53 mutant) lung cancer cell lines were maintained in RPMI media (Sigma Aldrich, UK). All medium was supplemented with 10% FCS (normal (Invitrogen, UK) or dialysed (Autogene Bioclear, UK)), 1% pen/strep, 1% L-Glutamine (All Invitrogen, UK).

### Xenograft Models

6-8 week old female SCID mice were implanted with 2×10⁶ HCT116+/+ human colorectal adenocarcinoma cells into each flank. HCT116 cells in a log phase of growth were harvested, washed in PBS and resuspended in HBSS. They were mixed with equal volumes of matrigel to give a final concentration of 5×10⁶ cells/ml. Mice were randomly separated into treatment groups on day 5 after implantation and treated with different doses of chemotherapy. 5-Fu (70 mgs/kg), CPT11 (70 mgs/kg) or saline control solution and the mice have been sacrificed at different time points (24 and 48 hrs after injections). All drugs were administered through a bolus injection. Animals were sacrificed at various time points and tumours were removed for analysis

### Microarray Analysis

Approximately 10 μg total RNA was isolated from tumour cells and was used as the starting material for preparation of probes. The microarray analysis was carried using an Affymetrix U133 plus 2.0 GeneChip®. Probes were prepared as per the manufacturers recommendations.

After RNA extraction, samples were reverse transcribed into cDNA which was then purified on a column prior to labelling. The probes were then amplified and labelled using Oligo(dT)-primed in vitro transcription generating high-yield, biotinylated targets from the 3′-end. The cRNA was fragmented to obtain optimal assay sensitivity and then subjected to quality control to confirm that fragment sizes range from 35-200 nucleotides. cRNA was quantified on a spectrophotometer and the quality of fragmented cRNA checked on a bioanalyser. For the next stage the fragmented cRNA was hybridised to the array for 16 hours at 45° C. Following this the array was washed and stained with streptavidin-phycoerythrin (SAPE) using a fluidics station and scanned using a GeneChip® Scanner 3000. Stained images were then analysed.

Initially the data was scaled using the Affymetrix® GCOS (Genechip® Operating System) software, to assess quality metrics. The data was then normalized against a control sample. After normalisation the data was filtered removing all genes where the noise level obscured signal and were fold change was greater than 2-fold. Finally a confidence filter where the t-test p-value were used to filter the genes to derive lists of statistically robust data.

Each treatment type and timepoint was carried out in triplicate and statistics and filtering were applied to the whole data set from each condition.

### Chemotherapy Treatment

a—Cell Culture

Cells in a log phase of growth were seeded into T75 flask at ˜20% confluence and incubated overnight to allow adherence to the plate. Wells were treated with CPT11 (Irinotecan) and 5-Fu (Fluorouracil) at 7.5 μM concentration for 48 hours. Chemotherapy was substituted with saline in control flasks. After different time exposure to chemotherapy the cells were harvested, washed 3 times in PBS and total RNA was isolated using the RNA TAT-60 reagent according to the manufacturer instructions.

Reverse transcription was performed with 2.5 μg of RNA using a High Capacity cDNA Archive kit (Applied Biosystem) according to the instruction of the manufacturer.

b—Organs

For in vivo toxicity study, mice were inoculated with 2×106 HCT116+/+ human colorectal adenocarcinoma cells into each flank. Mice were randomly separated into treatment groups on day 5 after implantation and treated or not with 5Fu (15 mg/kg daily or 70 mg/kg twice weekly). All drugs were administered through a bolus injection. Animals were sacrificed at various time points and organs and tumor were removed for analysis.

Semi quantitative RT-PCR

Semi quantitative RT-PCR was performed using a PTC 225 Gradient Cycler (MJ Research Incorporated. The PCR mixture, in a final volume of 25 μL, contains 12.5 μL of 2× Biomix (Bioline, UK), 2 μL of primers (10 μmol/L), 1 μL of cDNA and 9.5 μL of dH₂O). PCR conditions were initial denaturation step of 95 C for 10 minutes, followed by 35 cycles of 95° C. for 30 sec for denaturation; as annealing step 55° C. for 30 sec; and extension at 72° C. for 90 sec, with a final extension of 72° C. for 10 minutes. 5 μl of amplified product reactions was loaded onto a 1.5% agarose gel (0.001% ethidium bromide) which was ran at 90V for 30 to 40 minutes prior to analysis on a UV box. A Beta-actin control PCR amplification was performed for each cDNA to check the level of cDNA charged in the PCR mix.

For Example 2, Total RNA was isolated from cells following the RNA STAT-60 manufacturer's protocol (Biogenesis, Poole, U.K.). RT-PCR was performed with 1 μg of total cell RNA using a Promega ImProm-II™ Reverse Transcription System (Promega, Southhampton, UK). PCR was performed using primer pairs specific for human AREG (Forward 5′-TTTTTTGGATCCCTCGGCTCAGGCCATTATGCTGCT-3′(SEQ ID No:19), Reverse 5′-TTTTTTAAGCTTTACCTGTTCAACTCTGACTG-3′ (SEQ ID No:20)), human HB-EGF (Forward 5′-TTTCTGGCTGCAGTTCTCTCGGCACT-3′(SEQ ID No:21), Reverse 5′-CCTCTCCTATGGTACCTAAACATGAGAAGCCCC-3′(SEQ ID No:22)), human GAPDH as a control (Forward 5′ACCACAGTCCATGCCATCAC-3′(SEQ ID No:23), Reverse 5′ TCCACCACCCTGTTGCTGTA-3′(SEQ ID NO:24)) and human Beta-actin as a control (Forward 5′-ATCTGGCACCACACCTTTACAATGAGCTGCG-3′(SEQ ID No:25), Reverse 5′-CGTCATACTCCTGCTTGCTGATCCACATCTGC-3′(SEQ ID No:26)).

The PCR products were separated on 1.5% agarose gel and visualized by ethidium bromide staining.

### Western Blotting

a—Cell Lysis

HCT 116, RKO, HT 29 and H630 are human colorectal carcinoma cell lines. After different time exposure to chemotherapy the cells were harvested, and washed in 1×PBS. The cell pellet was then lysed in a suitable amount of 1×RIPA lysis buffer (150 mM NaCl, 10 mM Tris at pH 7.2, 0.1% SDS, 1.0% triton X-100 and 5 mM.EDTA) supplemented with protease inhibitors. The cell lysate was briefly vortexed, incubated on ice for 10 min and then centrifuged at 12,000 rpm to remove cell debris. Following centrifugation, the lysate supernatant was removed to a fresh eppendorf tube.

b—Quantitation of Whole Cell Lysates (WCL)

Protein concentrations were assayed using the BCA Protein Assay (Pierce) according to the manufacturer's instructions. The absorbance of each sample at 620 nm was assayed using a microplate reader. A standard BSA curve was plotted for each experiment and the protein concentration of each sample calculated.

c—Preparation of Whole Cell Lysate (WCL) protein Samples

To each sample an equal volume of 5× Western sample buffer and 10% of the final volume of β-mercaptoethanol (Sigma) was added. The samples were denatured at 95° C. for 5 minutes and placed on ice prior to loading onto SDS polyacrylamide gel (SDS PAGE). 10 μl of pre-stained protein molecular weight marker was loaded into one well of the gel.

d—Electro-transfer of Proteins to Polyvinylidene Flouride (PVDF) Membrane

After electrophoresis the gel was placed in 1× Western transfer buffer. A piece of PVDF (Millipore) was soaked in 100% methanol for 30 seconds then washed with deionised H₂O and equilibrated in 1× transfer buffer.

The above were then assembled into a Trans-blot SD semi-dry transfer cell (Bio-Rad) as follows: One piece of blotting paper soaked in transfer buffer, the PVDF membrane, followed by the gel, then one piece of blotting paper soaked in transfer buffer. The proteins were then electrophoresed onto the membrane at 20V for 90 min.

e—Immunoblotting (Western Blotting)

Following transfer, the PDVF membrane was washed three times for 10 minutes with 1×PBS/0.1% Tween before being blocked for 1 hour in 1×PBS/5% milk. Once blocked the membrane was incubated with the appropriate primary antibody at the relevant dilution, in 1×PBS/0.1 Tween/5% milk for 1 hour. Following incubation the membrane was washed three times with 1×PBS/0.1% Tween before being probed with the appropriate secondary antibody (Bio-Rad) at a dilution 1:5000 in 1×PBS/5% milk for 1 hour. The membrane was subsequently washed three times with 1×PBS/0.1% Tween for 10 minutes each before visualisation using Super Signal detection method (Pierce), as described by the manufacturers. Protein bands were detected by exposure to autoradiograph which was subsequently developed. If detection of a second protein was required from the same immunoblot, the membrane was placed in western stripping buffer, incubated for 30 min in a 50° C. rocking incubator. Following membrane stripping it was washed in 1×PBS/0.1% Tween, 5 times, for 10 min periods. The membrane was reprobed, as before with the appropriate antibodies.

### RNA Interference

AREG, HB-EGF and Control siRNAs and Dharmafect 4 transfection reagent were obtained from Dharmacon, (Lafayette, Colo., USA).

For HB-EGF, the siRNAs used had the following sequences:

| | |
|---|---|
| 1 Sense sequence | |
| GAAAAUCGCUUAUAUACCUUU | (Sequence ID No: 1) |
| 1Anti-sense sequence | |
| AGGUAUAUAAGCGAUUUUCUU | (Sequence ID No: 2) |
| 2 Sense Sequence | |
| UGAAGUUGGGCAUGACUAAUU | (Sequence ID No: 3) |
| 2 Anti-sense sequence | |
| UUAGUCAUGCCCAACUUCAUU | (Sequence ID No: 4) |
| 3 Sense sequence | |
| GGACCCAUGUCUUCGGAAAUU | (Sequence ID No: 5) |
| 3 Antisense sequence | |
| UUUCCGAAGACAUGGGUCCUU | (Sequence ID No: 6) |
| 4 Sense Sequence | |
| GGAGAAUGCAAAUAUGUAUU | (Sequence ID No: 7) |
| 4 Anti-sense Sequence | |
| UCACAUAUUUGCAUUCUCCUU | (Sequence ID No: 8) |

For AREG, the siRNA sequences used had the following sequences:

| | |
|---|---|
| 1 Sense Sequence | |
| UGAUAACGAACCACAAAUAUU | (Sequence ID No: 9) |
| 1 Anti-Sense Sequence | |
| UAUUUGUGGUUCGUUAUCAUU | (Sequence ID No: 10) |
| 2 Sense Sequence | |
| UGAGUGAAAUGCCUUCUAGUU | (Sequence ID No: 11) |
| 2 Anti-sense Sequence | |
| CUAGAAGGCAUUUCACUCAUU | (Sequence ID No: 12) |
| 3 Sense Sequence | |
| GUUAUUACAGUCCAGCUUAUU | (Sequence ID No: 13) |
| 3 Anti-Sense Sequence | |
| UAAGCUGGACUGUAAUAACUU | (Sequence ID No: 14) |
| 4 Sense Sequence | |
| GAAAGAAACUUCGACAAGAUU | (Sequence ID No: 15) |
| 4 Anti-sense Sequence | |
| UCUUGUCGAAGUUUCUUUCUU | (Sequence ID No: 16) |

Cells were seeded at 5000 cells per well in a 96 well plate or 5×10⁵ cells per well in a 6 well plate. The cells were cultured for 24 hours before transfection. The siRNA was made up to 100 nM in serum free DMEM and left for 5 minutes at room temperature. The Dharmafect transfection reagent was also made up in the serum free DMEM and incubated for 5 minutes at room temperature. The transfection reagent was added to the siRNA and incubated at room temperature for 20 minutes. The media was removed from the plate wells and antibiotic free DMEM was added to the wells. After 20 minutes the siRNA was added dropwise to the wells. The plates were incubated at 37° C. for 48 hours.

### MTT Assay

Cell viability was assessed by 3-(4,5-dimethylthiazol-2-yl)-2,5-diphenyltetrazolium bromide (MTT, Sigma) assay (Mosmann, T. 1983. J. Immunol. Methods 65:55-63). To assess chemotherapy/siRNA interactions 5000 cells were seeded per well on 96 well plate. After 24 hours cells were transfected with siRNA and treated with various chemotherapeutic agents at different concentrations. After 48 hours MTT (1.0 mg/ml) was added to each well and cells were incubated at 37° C. for 2 hours. The culture media was removed and formazan crystals were reabsorbed in 200 μl DMSO. Cell viability was determined by reading the absorbance of each well at 570 nm using a microplate reader (Tecan Sunrise, Biorad, UK).

### Cloning of Amphiregulin (AREG)

The DNA sequence encoding the amphiregulin protein was amplified by PCR from a cDNA library using gene-specific primers. The AREG gene was cloned into the bacterial expression vector pET100 allowing the incorporation of a hexahistidine tag onto the N-terminus of the recombinant protein. This construct was then used to transform competent TOP10F′ E. coli cells (Invitrogen). Positive transformants were selected by colony PCR using vector-specific primers flanking the multiple cloning site.

### Expression of Recombinant AREG Protein

The positive clones were propagated overnight at 37° C. in 5 mls of Luria-Bertani (LB) broth supplemented with 50 μM ampicillin. A 300 μl aliquot of this culture was retained for inoculation of secondary cultures and the remainder of the sample was miniprepped using the Qiagen miniprep kit and the sequence verified by DNA sequencing.

Three secondary cultures were inoculated to allow visualisation of protein expression. The cultures were induced with IPTG (final concentration 1 mM) when the cultures had an OD of 0.2, 0.5 and 1.0 (A₅₅₀) respectively and then left for 4 hrs at 37° C. The cells were then harvested by centrifugation at 4000 rpm for 15 mins and the pellet resuspended in 1 ml of PBS/0.1% Igepal supplemented with 1 μl of lysonase. Samples were then analysed by SDS-PAGE and western blotting to confirm expression of the protein. The SDS-PAGE gel was stained overnight in coomassie blue and destained the following day.

The recombinant AREG protein was then expressed in 500 mls of LB broth supplemented with ampicillin, using the secondary culture as an inoculant and induced with IPTG once the culture had reached the optimal optical density. The culture was centrifuged at 5000 rpm for 15 mins and the pellet retained for protein purification.

### Protein Purification

The induced recombinant protein was solubilised in 50 mls of 8 M urea buffer (480 g Urea, 29 g NaCl, 3.12 g NaH2PO4 (dihydrate), 0.34 g Imidazole) overnight. The solution was centrifuged at 6000 rpm for 1 hr, after which the supernatant was filtered using 0.8 μm gyrodisc filters before purification.

The protein was purified by its N-terminal hexahistidine tag and refolded using on-column refolding by immobilized metal affinity chromatography. Chelating hi-trap columns (Amersham Biosciences) were charged using 100 mM nickel sulphate before attachment to the Aktaprime. Refolding takes place by the exchange of the 8 M urea buffer with a 5 mM imidazole wash buffer (29 g NaCl, 3.12 g NaH2PO4 (dihydrate) 0.34 g Imidazole, pH 8.0) and elution of the protein using a 500 mM imidazole elution buffer (29 g NaCl, 3.12 g NaH₂PO₄ (dihydrate), 34 g Imidazole). The elution profile of the purified recombinant protein was recorded and can be seen in FIG. 16.

The eluted fractions were subjected to SDS-PAGE analysis to confirm recombinant protein presence in eluted fractions. The gels were stained with coomassie blue overnight and subsequently destained to determine the fractions containing the AREG protein.

### Antibody Generation

The refolded protein was used as an immunogen to generate monoclonal antibodies. Five BALB/C mice were immunized at three weekly intervals with 150 μg of purified recombinant protein and the antibody titre was analysed after boosts three and five. A test bleed was taken from each animal and tested at 1:1000 dilutions in western blotting against 100 ng of antigen. Blots were developed using 3,3′-diaminobenzidine (DAB).

After the fifth boost, the spleen was removed from the mouse and the antibody producing B cells were fused with SP2 myeloma cells following standard protocols. Eleven days after the hybridoma fusion, the plates were examined for cell growth. Clones were screened by ELISA against recombinant protein and selected positive hybridomas were cloned twice by limiting dilution.

### ELISA

The monoclonal antibodies were screened by ELISA to determine which clones should be expanded. Maxi Sorb 96 well plates were coated with recombinant antigen by adding 100 μl of coating buffer (Buffer A: 0.42 g sodium bicarbonate/100 μl H₂O, Buffer B: 0.53 g sodium carbonate/100 μl H₂O, pH 9.5) containing the screening antigen to each well (100 ng/well). A control antigen was also used to eliminate non-specific clones. The plates were incubated at 37° C. for 1 hr to allow the antigen to bind to the well and then blocked for 1 hr at room temperature by adding 200 μl PBS/3% BSA to each well.

The blocking solution was removed from the plates and 100 μl of hybridoma supernatant was added to a positive antigen and a control antigen well. The screening plates were incubated with supernatant for 1 hr on a rocker at room temperature. The plates were washed three times with PBS-T, after which 100 μl of goat anti-mouse HRP conjugated secondary antibody (1:3000) was added to each well and incubated for 1 hr at room temperature. The plates were washed three times with PBS-T and 100 μl of 3,3′,5,5′-tetramethylbenzidine (TMB) was added to each well and incubated for 5 mins at 37° C. Positive wells were indicated by a colour development and the reaction was stopped by addition of 50 μl 1M HCL. Plates were read by a spectrophotometer at 450 nm and samples displaying a positive reading in the screening well (+) with a negative reading in the control well (−) were chosen for further work. The cells from the original wells were transferred into a 24 well plate and grown up.

### Western Blotting

The supernatants from the hybridoma cell lines were analysed by western blotting to determine the ability of the monoclonal antibodies to detect both recombinant AREG and endogenous native AREG protein in a range of cancer cell lines, which are representative of AREG expression in cancer. Aliquots of HCT116 and HT29 whole cell lysates (˜30 μg/ml) or recombinant AREG protein were separated by SDS-PAGE and transferred onto Hybond-C Extra nitrocellulose membrane (Amersham Biosciences). The membrane was blocked by incubation in PBS/5% marvel for 1 hr at room temperature, after which it was rinsed briefly in PBS. The monoclonal antibodies were used at a 1:500 or 1:250 dilutions in PBS and incubated on the membrane overnight at 4° C. while gently rocking. The blot were then rinsed three times with PBS/1% marvel and 0.1% Tween-20 and then incubated with the goat anti-mouse HRP conjugated secondary antibody at a 1:3000 dilution for 1 hr at room temperature while shaking. The blots were then rinsed three times with the PBS/1% marvel and 0.1% Tween-20 solution, followed by a short rinse in PBS. The blots were incubated with ECL plus substrate (Amersham Bioscicences) for 5 mins at room temperature prior to analysis on the Kodak imager.

### Flow Cytometry Analysis

HCT116 or H460 cells were treated for 48 hours with or without 2.5 μM irinotecan. After 48 hours cells were washed in PBS and blocked for 20 minutes in Normal Goat Serum. 5×10⁵ cells were incubated with AREG antibodies or isotype control for 2 hours and washed in PBS-T. The cells were incubated with a FITC conjugated goat anti-mouse antibody for 1 hour and washed in PBS-T before analysis on BD FACS canto.

### Results

### Example 1

A xenograft study was set up to examine the genetic response to 2 different chemotherapeutic drugs 24 and 48 hours after treatment. Each mouse was implanted with equal volumes of HCT116 cells and each condition was performed in triplicate. 4 groups of three mice were administered of 100 ul CPT-11 (70 mg/kg), 5-FU (70 mg/kg) or saline control. Tumours were then resected after 24 h (5-FU) & 48 h (CPT-11, 5-FU). Average mass of the tumours did not vary over control and drug treated groups.

RNA isolated from tumours in each of the 12 mice was subjected to microarray analysis to measure mRNA expression levels. Fold change values for drug treated against untreated control is presented. After 48 hours, the fold change values for AREG mRNA expression in 5FU treated against untreated controls was 2.1 with the fold change values for AREG mRNA expression in CPT11 treated against untreated controls being 2.2. The data was passed through stringent statistical filters and is considered statistically robust. The amphiregulin RNA was significantly upregulated greater than 2 fold relative to control.

Five other ErbB cognate ligands have also been found to be up-regulated by our micro-array analysis. TGF and HB-EGF protein showed up-regulation when treated by 5-FU. EREG protein showed up-regulation after 48 h treatment with both CPT-11 and 5-FU. BTC protein showed up-regulation in all 3 conditions. NRG3 was upregulated after 48 h treatment with both CPT-11 and 5-FU. The results are summarised in Table 1.

The genes were selected for further study as a potential target for antagonists. To validate the expression data observed in the microarray semi quantitive RT-PCR was carried out for the gene. RT-PCR was carried out on RNA extracted from colorectal cell lines (including HCT116, RKO, HT29 & H630) following exposure to a relevant range of chemotherapeutic treatments

Results for the selected target using Q-PCR validated the results observed in the microarray analysis. AREG upregulation was validated in RKO cell lines 48 h after treatment with CPT-11 and 5-FU and in HCT116 cells 48 h after treatment with 5-FU (FIG. 1).

To make a suitable target for an AREG inhibitor preferential upregulation should be observed in tumour tissue when compared to other vital organs. For this experiment the inventors have used mouse homologues of the targets and examined regulation in mice organs, for the gene. None of the targets analysed displayed upregulation in the mouse organs examined which suggests the chemotherapeutic treatment has a more acute affect on expression in cancer cells than stable tissue.

To show that target upregulation observed at the mRNA level was mirrored at the protein level western blot analysis was performed. AREG protein expression in RKO and HCT116 p53 wild type colorectal cancer cell lysates was analysed following a 48 hour CPT11 or 5FU treatment. Western blots were probed using an anti-AREG antibody. Enhanced AREG expression was observed following CPT11 treatment with both the HCT116 and RKO cell lines (FIG. 2).

Confocal microscopy was used to analyse the in vitro effects of CPT-11 treatment (24 h and 48 h) of HCT116 cells on AREG expression levels. The inventors observed increasing levels of expression at each time point when compared to untreated controls (FIG. 3).

FIG. 4 illustrates analysis of AREG protein expression in H630 p53 mutant colorectal cancer cell lysates following a 48 hour CPT11 treatment. Western blots were probed using an anti-AREG antibody. Enhanced AREG expression was observed following chemotherapy (A and B illustrate two separate experiments) when compared to controls. This data demonstrates that CPT-11 (or indeed analogues or metabolites thereof) in combination with an ErbB cognate ligand (as shown to be upregulated) can be used for the treatment of p53 mutant cancers.

FIG. 5 illustrates analysis of AREG and beta actin RNA expression in H460 lung cancer cells with/without chemotherapy (either a 48 hour CPT11 treatment or 5-Fu treatment). RNA levels were analyzed following 35 cycle of PCR to determine relative differences in expression between treated and untreated samples. This data shows an enhanced expression of AREG following both CPT-11 and 5-Fu challenge.

TABLE 1
Result of microarray analysis performed on 5FU & CPT-11
treated HCT116+/+ cells (In Vivo) for the different
members of the EGF-family protein

| Gene | 24 h 5-FU | 48 h 5-FU | 48 h CPT-11 |
|---|---|---|---|
| EGF | −2.4 | −3.8 | −2.5 |
| (Epidermal | | | |
| Growth factor) | | | |
| TGF alpha | 1.3 | 2.2 | −2.1 |
| (Transforming | 1.8 | 1.0 | −1.2 |
| Growth factor | | | |
| alpha) | | | |
| BTC | 1.1 | 1.9 | 2.4 |
| (Betacellulin) | | | |
| HB-EGF | 1.6 | 1.6 | 1.0 |
| (Heparin- | 3.1 | −1.1 | −1.3 |
| Binding EGF- | | | |
| like growth | | | |
| factor) | | | |
| EREG | −1.5 | 1.9 | 1.5 |
| (Epiregulin) | 1.0 | 2.1 | 1.5 |
| NRG1 | N | 0 | T |
| NRG2 | −2.1 | −1.5 | −1.5 |
| | −2.5 | −4.7 | 1.5 |
| NRG3 | −2.1 | 1.6 | 1.2 |
| NRG4 | −1.1 | −1.8 | 1.2 |

This chemotherapy induced up-regulation of AREG has been observed at both the mRNA and protein level using p53 wt and mutant colorectal cancer cell lines (HCT116+/+; RKO+/+ and H630). AREG has also been shown to be up-regulated at the mRNA level in H460 lung cancer cells and MDA breast cancer cells which indicates that this effect may be observed over a range of AREG expressing cancers.

As seen from the molecular analysis, these proteins are selectively expressed after chemotherapy treatment in different carcinoma cell lines. This indicates that cancer cells, as a response to a chemotherapy challenge, over-express six different growth factors of the same family. This response seems to be a way used by the cancer cells to overcome chemotherapy insult. By selectively targeting these proteins, their role in cancer cell survival may be at least reduced and at best inhibited, which may lead to a reduction in tumour growth. Furthermore, the simultaneous targeting of two or more over expressed ligands (by an antagonist molecule like an antibody) may provide a useful therapeutic strategy.

### Example 2

### Chemotherapy Induced AREG Up-regulation in Colorectal and Breast Cancer Cell Lines

AREG up-regulation was further validated in several carcinoma cell lines. In human HT29 colorectal cancer cells and human HCT116 colorectal cancer cells AREG mRNA up-regulation was observed after treatment with IC₅₀ dose of CPT 11 (FIGS. 6A and 6B). Moreover, after treatment with IC₅₀ dose of 5-FU in human MDA-MB231 breast carcinoma cell line up-regulation of AREG mRNA was shown (FIG. 6C).

### Silencing of AREG and HB-EGF in Cancer Cells

siRNA potently down-regulated expression of AREG (FIG. 7A) and HB-EGF (FIG. 7B) in HCT116 colorectal cell line in comparison to untreated cells, mock transfection and control siRNA. In FIG. 9 and FIG. 11 respectively, AREG knockdown is also shown in HT29 colorectal cancer cells and in MDA-MB231.

Synergistic Attenuation of Cell Growth After Treatment with siRNA and Chemotherapy in Colorectal Cancer

Following confirmation of AREG and HB-EGF silencing by siRNA, MTT assays were performed to investigate the effect of down-regulation of these two genes on cell growth.

AREG siRNA alone, HB-EGF siRNA alone and monotherapy of CPT-11 had no significant effect on cell viability compared to untreated cells, mock transfection and control siRNA. However, co-treatment of HCT116 with AREG siRNA and CPT-11 resulted in synergistic decreases in cell viability. The same effect was observed when AREG siRNA was replaced with HB-EGF siRNA (FIG. 8).

In another colorectal cancer cell line, HT29 similar results were obtained as those observed with HCT116. AREG siRNA alone and control siRNA alone had no significant effect on the growth of the cells. The combination of AREG siRNA and CPT-11 had a synergistic effect on cell viability resulting in the decrease of cell growth (FIG. 10).

Collectively these results indicate that down-regulation of AREG/HB-EGF expression in combination with chemotherapy had a significant effect on the attenuation of cell growth in colorectal cancer.

Synergy between silencing of AREG and treatment with chemotherapy led to an attenuation of cell growth in breast cancer

Following transfection with control siRNA alone, transfection reagent alone (mock) and chemotherapy treatment, a 20% reduction in cell growth was observed. A further 23% decrease was observed when cells were transfected with AREG siRNA alone. Treatment with varying doses of 5-FU (2.5-6 μM) showed similar results to that of AREG siRNA alone. However, treatment with 7.5 μM 5-FU in combination with AREG siRNA lead to a further 20% reduction in growth (60% overall reduction in growth in comparison to the untreated, FIG. 12).

When siRNA experiments and MTT assays were performed using a combination of AREG and HB-EGF the inventors surprisingly observed a marked reduction in cell growth. Experiments were performed in the MDA-MB231 breast cancer cell line, to assess if knocking down AREG and HB-EGF had any affect on cell viability in breast cancer cells.

Remarkably, co-silencing AREG and HB-EGF resulted in a significant decrease on cell viability compared to controls. A decline in cell growth of ˜75% was observed when MDA-MB231 cells were co-treated with target siRNA compared to controls FIG. 13.

### Development of AREG Specific Monoclonal Antibodies.

A panel of murine monoclonal antibodies were raised against recombinant human amphiregulin (FIG. 14-16). They were characterised by ELISA, western blotting (whole cell lysates from colorectal cell lines HCT116 and HT29) and confocal microscopy analysis for demonstration of specific recognition of AREG (FIG. 17-22).

### Up-regulation of AREG as Shown by FACS Analysis Using AREG Monoclonal Antibodies on Colorectal Cancer and Lung Carcinoma Cell Lines.

Flow cytometry analysis of HCT116 cells and H460 cells shows cell surface recognition of AREG when assessed using the Anti AREG monoclonal antibodies (FIG. 23 shows FACS analysis of HCT116 cells treated with AREG clones 4G5 and 6E11 and an isotype control). Furthermore, cells treated with 2.5 μM irinotecan for 48 hours prior to analysis showed up to a 40% increase in the cell surface expression of AREG. The Anti-AREG clone 6E11 1E9 detected a 20% increase in AREG expression in HCT116 cells after treatment with irinotecan (FIG. 24).

FACS analysis has also been carried out on the lung carcinoma cell line H460. Two clones namely 3H5 and 3F8 both detected AREG expression on the surface and up-regulation in expression levels after treatment with 2.5 μM irinotecan (FIG. 25).

These results demonstrate that the inventors' panel of AREG monoclonal antibodies recognise the AREG protein on the surface of the HCT116 cells.

Attenuation of Cell Growth in Cancer Cells after Treatment with AREG Monoclonal Antibodies.

MTT assays were performed on cancer cell lines, MDA-MB231 breast cancer cells (FIG. 26 & FIG. 27) and HCT116 colorectal cancer cells (FIG. 28) to ascertain the effect of AREG monoclonal antibodies on cell growth. The activities of different clones were screened on the MDA-MDB231 cell line with several showing ˜40% reduction in cell growth when compared to untreated cells. In HCT116 cells a more pronounced effect was observed with ˜60% reduction in the cell viability observed with clones 4G5 and 6E11. FIG. 29 shows similar effects on cell growth in the MDA-MB231 breast cancer cells with AREG clone 6E11 1E92D8 (which has been shown to be the same clone as 6E11 1E9 106). FIGS. 30 and 31 shows the effect of AREG antibodies in the HCT116 cell line. A 65% decrease in cell viability is observed in FIG. 30 and FIG. 31 shows a 50% decrease in cell growth. FIG. 32 shows the effect of the AREG antibody 6E11 1E9 106 in the lung cancer H460 cell line, comparing the effect against a isotypic control antibody and showing that the antibody significantly attenuates cell viability of the lung cancer cells. Together these results show that the AREG monoclonal antibodies have a significant effect on the cell viability of AREG expressing cancer cells including colorectal, lung and breast carcinoma.

Synergistic Attenuation of Growth on Colorectal Cancer Cells after Treatment with HBEGF siRNA and AREG Antibodies.

The effect of down-regulation of the HBEGF siRNA in combination with an AREG antibody was investigated by performing an MTT cell viability assay (FIG. 33). The HBEGF siRNA alone had a slight reduction on cell growth while the AREG antibody 6E11 1E92D8 had ˜50% reduction in cell growth. When the HBEGF siRNA and 6E11 1E92D8 was added in combination a further reduction in cell viability was observed. To see if this effect was synergistic the RI values as described by Kern 1988 and modified by Romanelli 1998 were calculated. The RI value is calculated as the ratio of expected survival (Sₑₓₚ defined as the product of the survival observed with drug A alone and the survival observed with drug B alone) to the observed cell survival(S_{obs}) for the combination of A and B. (RI=Sₑₓₚ/S_{obs}). Synergism is defined as RI>1. The RI value for 6E11 1E92D8 was approximately 2. However, the RI value for 6E11 1E9 106 was calculated as being above 5. Collectively these results show that the combined targeting of both HBEGF and AREG in the treatment of cancers associated with Erb ligands or EGF (including colorectal, breast and lung) results in a synergistic attenuation of cell growth.

The development of non-responsive tumours or chemotherapy resistant cancer remains a major obstacle to successful treatment. There is a clear need for tools which enable prediction of whether a particular therapy either single or combination will be effective against particular tumours. Moreover, there remains the need for new treatment regimes to increase the repertoire of treatments available.

Combined therapies have shown promising results by improving the response rates in patients by acting on the tumours through different mechanisms, the inventors' data suggests inhibitory molecules, for example antagonist antibodies, specific to AREG and HB-EGF used alone or in combination with chemotherapy could potentially be used to treat a wide variety of aggressive cancers including colorectal, lung and breast cancer.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1-21. (canceled)

22. A method of treating neoplastic disease in a subject comprising simultaneously, sequentially or separately, administering to said subject an effective amount of (i) an inhibitor of a first EGF and (ii) an inhibitor of a second EGF, wherein said first and second EGF are different EGFs.

23. The method according to claim 22, wherein the first EGF is HB-EGF and said second EGF are selected from the group consisting of AREG, TGF, EREG, BTC, and NRG3.

24. The method according to claim 22, wherein said inhibitor of said first EGF is an antibody which binds said first EGF or a nucleic acid molecule which inhibits expression of said EGF.

25. The method according to claim 22, wherein said inhibitor of said second EGF is an antibody which binds said second EGF or a nucleic acid molecule which inhibits expression of said EGF.

26. The method according to claim 22, wherein said inhibitor of said first EGF is a first siRNA and for said inhibitor of said second EGF is a second siRNA.

27. The method according to claim 22, wherein said second EGF is AREG.

28. The method according to claim 27, wherein the inhibitor is an anti EGF antibody.

29. The method according to claim 28, wherein the inhibitor of said second EGF is an antibody molecule which comprises a variable region having the amino acid sequence of Sequence ID No: 27 and a variable region having the amino acid sequence of Sequence ID No: 28.

30. The method according to claim 22, further comprising simultaneously, sequentially or separately administering to said subject an effective amount of (iii) a chemotherapeutic agent.

31. The method according to claim 30, wherein the chemotherapeutic agent is at least one selected from the group consisting of antimetabolites, topoisomerase inhibitors, alkylating agents, anthracyclines, and plant alkaloids.

32. The method according to claim 31, wherein the chemotherapeutic agent is selected from the group consisting of CPT-11 and 5FU.

33. A method of treating neoplastic disease in a subject comprising simultaneously, sequentially or separately, administering to said subject an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

34. The method according to claim 33, wherein said topoisomerase inhibitor is CPT-11 or SN-38.

35. The method according to claim 33, wherein said EGF is AREG and said EGF inhibitor is an anti-AREG antibody.

36. The method according to claim 35, wherein said anti-AREG antibody is an antibody molecule which comprises a variable region having the amino acid sequence of Sequence ID No: 27 and a variable region having the amino acid sequence of Sequence ID No: 28.

37. The method according to claim 33, wherein said EGF inhibitor is an siRNA.

38. The method according to claim 22, wherein said neoplastic disease is selected from the group consisting of colorectal cancer, breast cancer and lung cancer.

39. The method according to claim 22, wherein the neoplastic disease is a cancer comprising a p53 mutation.

40. A pharmaceutical composition comprising (i) an inhibitor of a first EGF and (ii) an inhibitor of a second EGF, wherein said first and second EGFs are different EGFs.

41. The composition according to claim 40, wherein said first EGF is HB-EGF and said second EGF is selected from the group consisting of AREG, TGF, EREG, BTC, and NRG3.

42. The composition according to claim 40, wherein said inhibitor of said second EGF is an antibody which binds said second EGF or a nucleic acid molecule which inhibits expression of said EGF.

43. The composition according to claim 42, wherein the inhibitor is an anti EGF antibody.

44. The composition according to claim 40, wherein said second EGF is AREG.

45. The composition according to claim 44, wherein the inhibitor of said second EGF is an antibody molecule which comprises a variable region having the amino acid sequence of Sequence ID No: 27 and a variable region having the amino acid sequence of Sequence ID No: 28.

46. The composition according to claim 40, wherein said inhibitor of said first EGF is a first siRNA and/or said inhibitor of said second EGF is a second siRNA.

47. A kit comprising, in combination for simultaneous, separate, or sequential use in the treatment of neoplastic disease, (i) an inhibitor of a first EGF and (ii) an inhibitor of a second EGF, wherein said first and second EGF are different EGFs.

48. The kit according to claim 47, wherein the first EGF is HB-EGF and said second EGF is selected from the group consisting of AREG, TGF, EREG, BTC, and NRG3.

49. The kit according to claim 47, wherein said inhibitor of said second EGF is an antibody which binds said second EGF or a nucleic acid molecule which inhibits expression of said EGF.

50. The kit according to claim 49, wherein the inhibitor is an anti EGF antibody.

51. The kit according to claim 47, wherein said second EGF is AREG.

52. The kit according to claim 51, wherein the inhibitor of said second EGF is an antibody molecule which comprises a variable region having the amino acid sequence of Sequence ID No: 27 and a variable region having the amino acid sequence of Sequence ID No: 28.

53. The kit according to claim 47, wherein said inhibitor of said first EGF is a first siRNA and/or said inhibitor of said second EGF is a second siRNA.

54. The kit according to claim 47, further comprising:
(iii) instructions for the administration of (i) and (ii) separately, sequentially or simultaneously.

55. A pharmaceutical composition for the treatment of cancer comprising an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

56. The pharmaceutical composition according to claim 55, wherein said topoisomerase inhibitor is CPT-11 or SN-38.

57. The pharmaceutical composition according to claim 55, wherein said EGF is AREG and said EGF inhibitor is an anti-AREG antibody.

58. The pharmaceutical composition according to claim 57, wherein the anti-AREG antibody is an antibody molecule which comprises a variable region having the amino acid sequence of Sequence ID No: 27 and a variable region having the amino acid sequence of Sequence ID No: 28.

59. The pharmaceutical composition according to claim 55, wherein said said EGF inhibitor is an siRNA.

60. A kit comprising, in combination for simultaneous, separate, or sequential use in the treatment of neoplastic disease, an effective amount of (i) an inhibitor of an EGF, wherein said inhibitor is a nucleic acid molecule which inhibits EGF expression or an anti EGF antibody, and wherein said EGF is HB-EGF or AREG, and (ii) a topoisomerase inhibitor.

61. The kit according to claim 60, wherein said topoisomerase inhibitor is CPT-11 or SN-38.

62. The kit according to claim 60, wherein said EGF is AREG and said EGF inhibitor is an anti-AREG antibody.

63. The kit according to claim 62, wherein the anti-AREG antibody is an antibody molecule which comprises a variable region having the amino acid sequence of Sequence ID No: 27 and a variable region having the amino acid sequence of Sequence ID No: 28.

64. The kit according to claim 60, wherein said EGF inhibitor is an siRNA.

65. A method of inducing and/or enhancing expression of a gene encoding an EGF protein in a cell or tissue comprising administering a topoisomerase inhibitor to said cell or tissue, wherein said EGF is selected from the group consisting of AREG, TGF, EREG, BTC, and NRG3.

66. An in vitro method for evaluating the response of tumor cells from a subject to the presence of a topoisomerase inhibitor to predict response of the tumor cells in vivo to treatment with the topoisomerase inhibitor comprising:
(a) providing a sample of tumor cells from a subject;
(b) exposing a portion of said sample to said topoisomerase inhibitor; and
(c) comparing expression of one or more genes encoding one or more EGFs₂ wherein said EGF is selected from the group consisting of AREG, TGF, EREG, BTC, and NRG3 in said portion of the sample exposed to said topoisomerase inhibitor with expression of said gene(s) in a control portion of said sample which has not been exposed to said topoisomerase inhibitor; wherein enhanced expression in the portion of sample exposed to said topoisomerase inhibitor is indicative of decreased sensitivity to said topoisomerase inhibitor.

67. A method of prognosis for evaluating a response of a patient to combination therapy comprising a topoisomerase inhibitor and an inhibitor of an EGF comprising:
(a) determining expression of a gene encoding an EGF in an in vitro sample containing tumor cells obtained from a subject prior to treatment with said chemotherapeutic treatment;
(b) determining expression of said gene encoding said EGF, wherein said EGF is selected from the group consisting of AREG, TGF, EREG, BTC, and NRG3, in an in vitro sample containing tumor cells obtained from a subject after treatment with said chemotherapeutic treatment; and
(c) comparing expression in (b) with expression in (a), wherein enhanced expression in (b) compared to (a) is indicative that the patient may benefit from combination therapy comprising a topoisomerase inhibitor and an inhibitor of said EGF.

68. The method according to claim 66, wherein the expression of said gene in the portion of sample exposed to said chemotherapeutic agent is considered to be enhanced if the expression is at least 1.5-fold that of the gene in the control portion of said sample which has not been exposed to said chemotherapeutic agent.

69. The method according to claim 65, wherein said gene encodes HB-EGF or AREG.

70. An antibody molecule comprising at least one of the CDRs of the 6E11 1E9 106 VH region having the amino acid sequence in Sequence ID No: 27 and/or at least one of the CDRs of the 6E11 1E9 106 VL region having the amino acid sequence in Sequence ID No: 28, wherein the antibody has binding specificity for AREG.

71. The antibody molecule according to claim 70, wherein the molecule comprises all three of the CDRS of the 6E11 1E9 106 VH region having the amino acid sequence in Sequence ID No: 27 and/or all three of the CDRS of the 6E11 1E9 106 VL region having the amino acid sequence in Sequence ID No: 28.

72. The antibody molecule according to claim 71, wherein the antibody molecule comprises a variable region having the amino acid sequence of Sequence ID No: 27.

73. The antibody molecule according to claim 71, wherein the antibody molecule comprises a variable region having the amino acid sequence of Sequence ID No: 28.

74. The antibody molecule according to claim 73, wherein the antibody molecule comprises a variable region having the amino acid sequence of Sequence ID No: 27 and a variable region having the amino acid sequence of Sequence ID No: 28.

75. An antibody molecule according to claim 74, wherein the antibody molecule is the 6E11 1E9 106 antibody, or a fragment thereof.

76. A pharmaceutical composition comprising the antibody molecule according to claim 70.

77. A method of treating neoplastic disease in a subject comprising administering to said subject the antibody molecule according to claim 70.

78. (canceled)
